(19) United States
(12) Patent Application Publication
(10) Pub. No.: US 2004/0191225 A1
(43) Pub. Date: Sep. 30, 2004

(54) Injection system

(76) Inventors: Jonathan H. Dinsmore, Brookline, MA (US); Douglas B. Jacoby, Wellesley, MA (US); Harout DerSimonian, Wellesley, MA (US)

Correspondence Address: Choate, Hall & Stewart, Exchange Place, 53 State Street, Boston, MA, 02109, US

(21) Appl. No.: 10/635,212

(22) Filed: Aug. 6, 2003

Related U.S. Application Data
(60) Provisional application No. 60/401,449, filed on Aug. 6, 2002.

(51) Int. Cl. A61K 45/00; A61M 31/00

(52) U.S. Cl. 424/93.7; 604/500

(57) ABSTRACT

Novel methods of injecting agents including cells into organs, tissues, or tumors using a side release needle and, optionally, a carrier that aids in retention of the cells at the injection site, or a tissue sealant or film to seal the injection site are provided. The use of side release needles and, optionally, a carrier, sealant, or film prevents the leakage of the injected agent back out of the injection site. Agents which may be injected using the inventive method include drugs, small molecules, peptides, proteins, polynucleotides, viruses, cells, etc. Any type of cells including myoblasts may be used in the invention. The cells may be injected into any organ including the heart, brain, pancreas, liver, etc. These injection methods may find use in tissue engineering, gene therapy, and tissue/organ repair. Kits with the side release needles used in carrying out the present invention are also provided.

### RELATED APPLICATIONS

This application claims the benefit of U.S. Provisional Application No. 60/401,449, filed Aug. 6, 2002, the entire teachings of which are incorporated herein by reference.

### BACKGROUND OF THE INVENTION

The local administration of an agent to the particular site where the agent is needed within the patient's body is useful in avoiding effects of the agent at other sites and in avoiding unwanted systemic side effects. By administering an agent to a particular tissue or organ, lower doses of the agent can be used since the agent is not delivered systemically. In the case of treating a neoplastic tumor, a chemotherapeutic agent may be delivered at the site of the tumor without the risk of effecting the patient's healthy tissues. Local administration is also important when a virus or polynucleotide to be used in gene therapy is being delivered so as to transfect only certain cells found in an organ or tissue. Administration to a particular site is also important when cells are delivered into a damaged area of tissue. The need to effectively deliver cells into a recipient's body for transplantation has become increasingly important as techniques have developed to culture cells with great potentials for differentiation and growth (i.e., precursor cells, myoblasts, stem cells) and to create cells which have had their genomes altered for gene therapy. Transplantation of cells has been used to treat diseases ranging from Parkinson's disease to diabetes to heart disease. The cells delivered may be derived from the recipient, a related donor, or another species than the recipient. One of the challenges of transplanting cells into an organ or area of the body is getting the cells into the correct location and having them grow, differentiate, and develop to become an integral part of the organ in which they are transplanted. Organs such as the heart and brain require that the transplanted cells be integrated into the existing network of cells to be fully functional. Cellular transplantation is particularly important in injured organs that can not repair themselves such as the heart and brain, and also in diseases where the cells of an organ are constantly being destroyed (e.g., type I diabetes).

As just one example of a disease in which cellular transplantation can be used, heart disease is the predominant cause of disability and death in all industrialized nations. Cardiac disease can lead to decreased quality of life and long term hospitalization. In addition, in the United States, it accounts for about 335 deaths per 100,000 individuals (approximately 40% of the total mortality) overshadowing cancer, which follows with 183 deaths per 100,000 individuals. Four categories of heart disease account for about 85-90% of all cardiac-related deaths. These categories are: 1) ischemic heart disease, 2) hypertensive heart disease and pulmonary hypertensive heart disease, 3) valvular disease, and 4) congenital heart disease. Ischemic heart disease, in its various forms, accounts for about 60-75% of all deaths caused by heart disease. In addition, the incidence of heart failure is increasing in the United States. One of the factors that renders ischemic heart disease so devastating is the inability of the cardiac muscle cells to divide and repopulate areas of ischemic heart damage. As a result, cardiac cell loss as a result of injury or disease is irreversible.

Human to human heart transplants have become the most effective form of therapy for severe heart damage. Many transplant centers now have one-year survival rates exceeding 80-90% and five-year survival rates above 70% after cardiac transplantation. Heart transplantation, however, is severely limited by the scarcity of suitable donor organs. In addition to the difficulty in obtaining donor organs, the expense of heart transplantation prohibits its widespread application. Another unsolved problem is graft rejection. Foreign hearts are poorly tolerated by the recipient and are rapidly destroyed by the immune system in the absence of immunosuppressive drugs. While immunosuppressive drugs may be used to prevent rejection, they also block desirable immune responses such as those against bacterial and viral infections, thereby placing the recipient at risk of infection. Infections, hypertension, and renal dysfunction caused by cyclosporin, rapidly progressive coronary atherosclerosis, and immunosuppressant-related cancers have been major complications.

### SUMMARY OF THE INVENTION

The present invention provides a system for injecting agents into a patient's body with minimal leakage of the injected agent from the injection site. To minimize leakage, the invention utilizes a needle having a side opening(s) rather than one with the opening at the tip of the needle and/or uses a sealant to seal the site after injection of the agent to be administered. In certain preferred embodiments, therapeutic agents, diagnostic agents, or prophylactic agents are administered using the inventive methods. These agents may include drugs, proteins, peptides, small molecules, polynucleotides, biological molecules, viruses, cells, etc. A particularly preferred agent to be delivered is cells. The agents may be injected into any organ, tissue, tumor (benign or malignant), site of injury or damage, site of malformation, or any other site in the patient's body. Preferably the organ or tissue is solid or substantially solid so as to provide some resistance to the injection of the agent to be delivered. In certain embodiments, the agent is injected into a site of injury in the target organ or tissue. Once the agent has been injected, preferably none or a minimal amount of leakage of the administered agent from the injection site is observed. Preferably, less than 50% of the agent leaks from the site, and more preferably less than 40%, 30%, 20%, 10%, 5%, 4%, 3%, 2%, or 1% of the agent leaks from the injection site. Most preferably no leakage from the injection site can be detected. The invention is particularly useful in injecting cells to be transplanted into organs such as cardiac muscle, brain, pancreas, liver, kidney, and skeletal muscle. The cells may be suspended in a carrier (e.g., collagen, gelatin, fibrin, methylcellulose, agarose, alginate, hyaluronic acid, etc.) that aids in retention of the cells at the injection site. In addition or alternatively, the injection site may be sealed with a tissue sealant (e.g., cyanoacrylate tissue adhesives, fibrin glue such as Tisseel®), a film (e.g., Seprafilm), or glue after injection to close the injection hole and prevent leakage of the injected agent.

The present invention may be used to treat a variety of conditions where injury to an organ results in damage that can be treated by delivery of a therapeutic agent. In certain embodiments, the organ damage can be treated by cellular transplantation. In particular, the invention provides a method for treating a condition characterized by damage to cardiac tissue comprising injecting skeletal myoblast cells into the site of myocardial injury using a side release needle such that the condition is thereby treated. The injections may be repeated so as to treat the cardiac condition.

In certain other embodiments, the invention may be used to deliver a drug to a specific site within the patient's body. For example, the inventive method may be used to deliver anti-neoplastic agents within a tumor mass so that the drug will have the maximum effect on the tumor and less of an effect on the surrounding tissues. The drug may be encapsulated or in such a form as to allow release of the active agent over time.

In another aspect, the invention provides a kit comprising a needle with a closed end and side openings. The kit may also include the agent to be delivered, for example, the drug, protein, peptide, polynucleotide, small molecule, biological molecule, virus, cells, etc. In certain embodiments, the kit may include cells to be transplanted, factors and media used in culturing cells for transplant, carriers (e.g., collagen, gelatin, extracellular matrix proteins, fibrin, methylcellulose, agarose, alginate, hyaluronic acid, etc.) that aid in the retention of cells, tissue sealants, tissue glues or adhesives (e.g., cyanoacrylate tissue adhesives, fibrin glues such as Tisseel®), sealing films (e.g., Seprafilm), solutions for sterilizing the injection site, suture material, equipment for extracting cells to be transplanted later, and/or a syringe. Preferably, any solutions, media, or equipment to be used in handling and injecting the agent to be delivered has been sterilized and packaged to prevent contamination.

### BRIEF DESCRIPTION OF THE DRAWINGS

FIG. 1 shows a Whitacre pencil point needle with a close-up of the end of the needle with its side opening.

FIG. 2 shows hematoxylin and eosin stain of injected heart. FIG. 2A shows the area of the heart injected with a standard 25G beveled needle, and FIG. 2B shows an adjacent region of the heart injected with a 25G Whitacre needle. Cells retained at the site of injection are marked by arrows. Many more cells were found in the area surrounding the injection with the Whitacre needle. For FIG. 2A, 400 μl of cell suspension containing 40 million cells were injected over 1 minute. For FIG. 2B, 400 μl of cell suspension containing 200 million cells was injected over 1 minute.

FIG. 3 shows results of injecting ischemically damaged sheep heart with skeletal muscle myoblasts using a side-port needle. Six weeks after injection the animals were sacrificed and the heart was stained with muscle-specific myosin immunostaning as shown in FIG. 3B and Trichrome stain as shown in FIG. 3B.

FIG. 4 shows results of injecting skeletal muscle myoblasts using a side-port needle into a human heart while the patient was undergoing surgery to implant a left ventricular assist device as a bridge to heart transplant surgery. Five days after injection, the patient died and his heart was stained with Trichrome stain as shown in FIGS. 4A (low magnification) and 4B (higher magnification).

### DEFINITIONS

“Angiogenesis”: “Angiogenesis” refers to the formation of new capillary vessels in the heart tissue into which the muscle cells of the invention are transplanted. Angiogenesis can occur as a result of the act of transplanting cells, as a result of the secretion of angiogenic factors from the transplanted cells, and/or as a result of the secretion of endogenous angiogenic factors from the organ into which the cells have been transplanted.

“Animal”: The term animal, as used herein, refers to human as well as non-human animals, including, for example, mammals, birds, reptiles, amphibians, and fish. Preferably, the animal is a mammal (e.g., a rodent, a mouse, a rat, a rabbit, a monkey, a dog, a cat, or a pig), most preferably a human. An animal may be a transgenic animal.

“Biological compounds”: “Biological compounds” are any chemical compounds found within a living organism. In certain embodiments, biological molecules may include DNA, RNA, polynucleotides, proteins, peptides, lipids, polysaccharides, oligosaccharides, and sugars.

“Cardiac myocyte”: “Cardiac mycocyte” refers to a muscle cell which is derived from cardiac muscle. Such cells typically have one nucleus and are, when present in the heart, joined by intercalated disc structures.

“Cell”: The term “cell” refers to any type of cell to be delivered using the inventive method. The cell may be derived from bacteria, fungi, yeast, plants, animals, mammals, or humans. If the cells is derived from a multi-cellular organism, it may come from any tissue or organ (e.g., skin, heart, skeletal muscle, smooth muscle, pancreas, brain, nerve, kidney, liver, stomach, intestines, etc.). The cell may be derived from the patient to whom they are to be delivered, from a related donor, from a family member, from a donor with similar MHC markers, from an unrelated donor, or from a donor of another species (e.g., a pig). The cell may be obtained from cell culture.

“Isolated”: The term “isolated” refers to a cell which has been separated from its natural environment. This term includes gross physical separation of the cell from its natural environment, e.g., removal from the donor. Preferably “isolated” includes alteration of the cell's relationship with the neighboring cells with which it is in direct contact by, for example, dissociation.

“Myocardial ischemia”: “Myocardial ischemia” includes a lack of oxygen flow to the heart which results in myocardial ischemic damage. As use herein, myocardial ischemic damage refers to damage caused by reduced blood flow to the myocardium. Non-limiting examples of causes of myocardial ischemia and myocardial ischemic damage include decreased aortic diastolic pressure, increased intraventricular pressure and myocardial contraction, coronary artery stenosis (e.g., coronary ligation, fixed coronary stenosis, acute plaque change (e.g., rupture, hemorrhage), coronary artery thrombosis, vasoconstriction), aortic valve stenosis and regurgitation, and increased right atrial pressure. Non-limiting examples of adverse effects of myocardial ischemia and myocardial ischemic damage include myocyte damage (e.g., myocyte cell loss, myocyte hypertrophy, myocyte cellular hyperplasia), angina (e.g., stable angina, variant angina, unstable angina, sudden cardiac death), myocardial infarction, and congestive heart failure.

“Patient”: A patient may be of any species. Patients may be humans, domesticated animals, dogs, cats, birds, pets, fish, hamsters, rats, gerbils, etc. In certain preferred embodiments, the patient is a human. The patient may or may not be suffering from illness at the time of treatment using the inventive method. For example, the inventive method may be used to deliver a prophylactic agent such as a vitamin or birth control agent. In other embodiments, the patient will be suffering from a disease such as cardiac disease, diabetes, Parkinson's disease, cancer, genetic defect, etc.

“Peptide” or “Protein”: According to the present invention, a “peptide” or “protein” comprises a string of at least three amino acids linked together by peptide bonds. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Polynucleotide” or “oligonucleotide”: Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyladenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-hydroxylribose, 2′-fluororibose, ribose, 2′-deoxyribose, and hexose), or modified phosphate groups (e.g., phosphorothioates and 5′-N-phosphoramidite linkages).

“Sealant” or “sealing”: “Sealing” refers to the use of a sealant to close an injection site or site of administration of an agent. The sealant prevents leakage of the administered agent from the site of delivery. Any material that can close a injection hole can be used as a sealant. Sealants may be glues, adhesives, or films. The sealing may be done concurrently with delivery of the agent or may be performed subsequent to administration. Examples of sealants include cyanoacrylate tissue adhesives, fibrin sealant such as Tisseel® (marketed by Baxter International Inc.), Seprafilm, polymers, proteins, etc. In certain embodiments, the sealant is used after the agent has been delivered using a needle with a side opening.

“Skeletal myoblasts”: “Skeletal myoblasts” are precurors of myotubes and skeletal muscle fibers. The term “skeletal myoblasts” includes satellite cells, mononucleate cells found in close contact with muscle fibers in skeletal muscle. Satellite cells lie near the basal lamina of skeletal muscle myofibers and can differentiate into myofibers.

“Small molecule”: The term “small molecule”, as used herein, refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of small molecules that occur in nature include, but are not limited to, taxol, dynemicin, and rapamycin. In certain other preferred embodiments, natural-product-like small molecules are utilized.

“Solid organ”: “Solid organ” refers to any tissue or organ within a patient's body. The inventive method may be used to deliver any agent including cells into the solid organ. The solid organ may be a normal organ (e.g., heart, pancreas, brain, liver, kidney, skeletal muscle, etc.) or an abnormal growth such as a benign or malignant tumor. A solid organ may have a lumen or space in it such as the small and large intestines or the lung. In certain embodiments, the solid organ is a tissue which will provide resistance to the introduction of additional matter such as cells or a liquid. In certain preferred embodiments, the solid organ is cardiac muscle.

“Stem cell”: “Stem cell” refers to any pluripotent cell that under the proper conditions will give rise to a more differentiated cell. Stem cells which may be used in accordance with the present invention include hematopoietic, neural, mesenchymal, gastrointestinal, muscle, cardiac muscle, kidney, skin, lung, and embryonic stem cells.

“Therapeutically effective amount”: The term “therapeutically effective amount” refers to the amount of an agent needed to elicit the desired biological response. In a preferred embodiment, the therapeutically effective amount of an agent is delivered using a minimum number of injection so as not to damage the target organ by using multiple injection; therefore, each injection should preferably result in the retention of a substantial portion of the agent being delivered. In the present invention, the agent can be drugs, small molecules, peptides, proteins, polynucleotides, biological molecules, viruses, and cells (e.g., stem cells, skeletal myoblasts, etc.). For example, in the case of an infection, the therapeutically effective amount of antibiotic is the amount necessary to clear the infection or kill all the organisms responsible for the infection. In the case of gene therapy, the therapeutically effective amount of polynucleotide (e.g., vector, artificial chromosome), virus, or cells is the amount necessary to correct the recipient's underlying genetic defect. In the case of tissue damage or degeneration, the therapeutically effective amount of cells is the amount necessary to improve the function or structure of the abnormal or damaged tissue. For example, in the transplantation of cells for cardiac injury, the therapeutically effective amount of cells is the amount necessary to improve the functioning of the heart by increasing cardiac output, increasing stroke volume, decreasing anginal symptoms, or improving cardiac status of the patient transplanted.

### DETAILED DESCRIPTION OF CERTAIN PREFERRED EMBODIMENTS

The invention in one aspect provides a method of delivering agents using a needle with a closed end and at least one side opening. Typically the agent is injected using the side-opening needle into an organ or a substantially solid tissue or tumor, rather than an opening, hole, negative space, or lumen. In certain embodiments, the injection site is a damaged or diseased area within an organ or tissue. The agents that can be delivered using the inventive method include drugs, small molecules, polynucleotides, proteins, biological molecules, antibodies, viruses, cells, etc. In one embodiment, the inventive method is used to deliver an agent such as a drug to a specific location within the patient's body, i.e., into a specific organ or tissue. In another embodiment, the method may be used to deliver cells for therapeutic purposes such as to restore and/or replace diseased, injured, scarred, or dead tissue. The method may also be used in gene therapy wherein the genomes of the cells or viruses to be delivered have been altered.

Needle

The needle used in carrying out the inventive method has an opening on the side of the shaft of the needle rather than at the end. The closed end may be a beveled tip, a curved tip, or a pencil point tip. The openings on the side are typically located near the tip of the needle. There may be one opening on the side of the needle, or there may be a series of side openings. For example, there may be two openings on opposite sides of the needle, or there may be a series of coaxial openings.

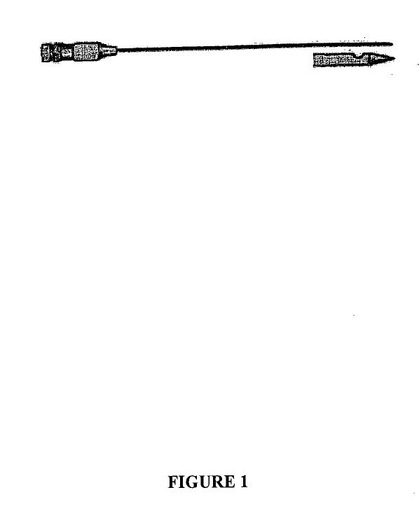

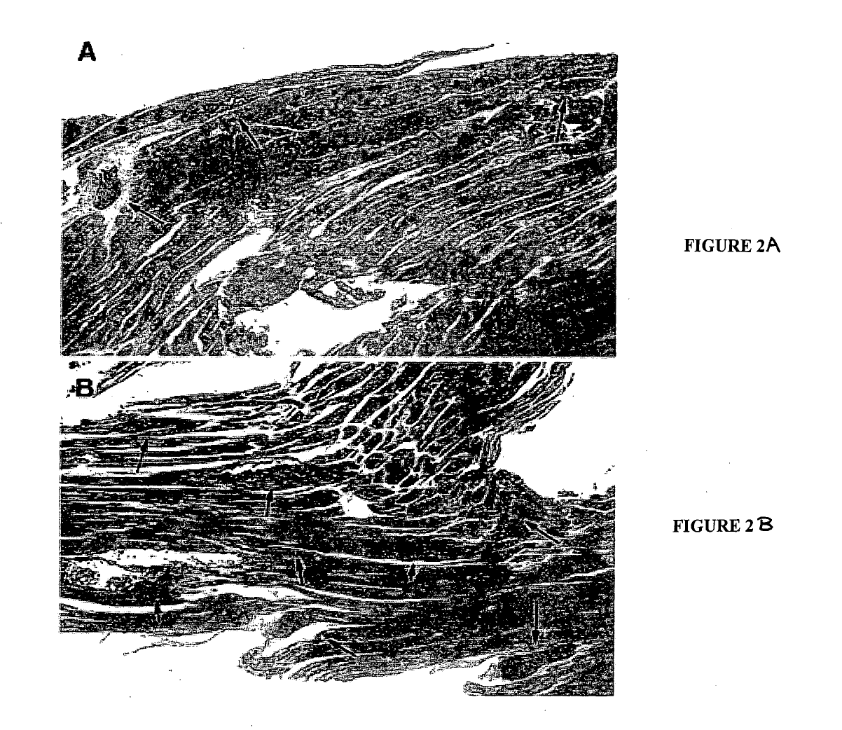

Needles that are particularly useful in the inventive method include spinal needles that are used to access the cerebrospinal fluid (CSF) of a patient (see, for example, U.S. Pat. No. 5,848,996, incorporated herein by reference). These spinal needles were initially designed for spinal anesthesia and lumbar puncture to prevent the leakage of CSF, which can result in post puncture headaches in some patients. These needles include the Whitacre needle as shown in FIG. 1 and the Sprotte needle (see, also, U.S. Pat. No. 5,848,996, issued Dec. 15, 1998, and U.S. Pat. No. 5,449,351, issued Sep. 12, 1995; each of which is incorporated herein by reference).

In order to prevent the creation of a large bore hole which would allow the injected agent egress, a needle with a small radius is preferred rather than a larger radius. The smaller radius corresponds to a larger gauge. Typically, the gauge of the needle used in the present invention will range from approximately 20 to approximately 30 gauge and probably more preferably approximately 25 gauge. The gauge of the needle will also be determined by the strength of the needle needed, the size of the agent (e.g., cells) to be injected, the viscosity of the agent, suspension of the agent, or solution of the agent to be injected, the delicacy of the organ or tissues to be penetrated, the control needed during the injection procedure, etc. For example, a needle having a larger gauge (e.g., 30 gauge) may be useful in delivering a small molecule, drug, or virus, but a smaller gauge needle may be needed to inject an agent with a larger size such as cells. The gauge of the needle will best be determined by the medical professional performing the method taking into account the various factors laid out above and using the best judgment of the professional and his experience doing similar procedures.

The length of the needle will depend on various factors surrounding the injection of the agent. These factors may include the organ being injected into, the depth of the site where the cells are to be delivered, the control of the needle needed to perform the injection, what tissues must be penetrated to get to the transplantation site, etc. Again, as with the gauge of the needle, the length of the needle is best determined by the medical professional performing the procedure. Typically, the length of the needle will be between ½″ and 7″, preferably between ½″ and 4″, and more preferably, between ½″ and 3″.

The needle may also have certain other characteristics designed for a particular use. For example, the size and shape of the side openings may depend on the cells to be delivered and the site at which they will be delivered. Also, the needle may be curved or kinked in order to provide easy access to a certain transplantation site. The pattern of openings on the shaft of the needle and the location of the openings vertically on the shaft may depend on the site and organ for transplantation.

Agents to be Delivered

Any agent that can be injected through a needle can be delivered using the inventive method. Typical agents might include drugs, small molecules, pharmaceutical agents, diagnostic agents, biological molecules, proteins, peptides, antibodies, polynucleotides, RNA, DNA, viruses, cells, and combinations thereof. Agents may range in size from small organic molecules to macromolecules such as DNA to intact cells. The agent to be delivered to the injection site may be therapeutic (e.g., chemotherapeutic drug, antibiotic), prophylactic (e.g., vaccine), or diagnostic (e.g., contrast agent for magnetic resonance imaging, labeled metabolite).

Drugs include any compound useful in the treatment or prevention of a disease. Many drugs have been approved by the Food and Drug Administration for the treatment of diseases in humans. In a particularly preferred embodiment, the drug is an antibiotic, anti-viral agent, anesthetic, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, nutritional agent, etc. A combination of drugs may be used in the present invention. The drug may also be delivered in various forms, for example, the drug may be encapsulated, or the drug may be in a time release form.

Agents to be delivered may also include biological molecules such as proteins, peptides, polynucleotides, and oligonucleotides. Examples of proteins or peptides include insulin, cytokines, growth factors, erythropoeitin, antibodies, antibody fragments, etc. Polynucleotides may be delivered for gene therapy and anti-sense therapy. The polynucleotides may include any of the following elements: open reading frames, promoters, enhancer regions, ribosomal binding sites, regulatory regions, splicing signals, introns, exons, etc.

In addition to drugs, small molecules, and biological molecules, the invention may be used to deliver viruses and cells. Particularly preferred viruses and cells are those that are therapeutic. Viruses with altered genomes may be used in gene therapy as vectors to introduce a foreign gene into the patient's cells. The viruses may be used to deliver a gene to correct a genetic defect in the patient's own genome. In certain embodiments, the viruses may be altered to lessen their antigenicity.

The inventive method may also be used to deliver cells. Any type of cell or mixture of cells may be transplanted using the inventive method. Cell types particularly useful in the present invention include cardiac muscle cells, skeletal muscle cells, beta-islet cells, hepatic cells, hematopoietic cells, neurons, fibroblasts, stem cells, etc. The cells may be at any stage of differentiation ranging from omnipotent embryonic stem cells to fully differentiated cells. Cells are chosen depending on the site of the transplantation and the nature of the defect or injury to be repaired. For example, an area of myocardium injured due to ischemic heart disease may be repaired by transplanting skeletal myoblasts or a mixture of skeletal myoblasts and fibroblasts. Preferably the cells have been purified to eliminate unwanted cells types or cells that would cause adverse reactions such as an immunological response. The cells may be purified by FACS sorting, immunological techniques, passage in cell culture, etc. Preferably the cells have been suspended in a medium for injection and transplantation. The cells may be suspended at concentrations for injection ranging from 1×10⁶ cells/ml to 1000×10⁶ cells/ml, more preferably 10×10⁶ cells/ml to 500×10⁶ cells/ml, even more preferably from 50×10⁶ cells/ml to 200×10 ⁶ cells/ml, and most preferably from 50×10⁶ cells/ml to 100×10⁶ cells/ml. The cells may be obtained from cell culture, from donors, from tissue and blood banks, from a relative of the recipient, or from the recipient himself. The cells may also be obtained from an animal that is not the same species as the recipient. The cells are typically provided as a homogeneous suspension of cells in medium or some other solution. The cells may be provided in an isotonic solution, or in the use of certain cell types such as myoblasts, the cells may be suspended in a hypertonic solution.

The cells may be obtained from a biopsy of tissue taken from another person, the recipient himself, or an animal of another species (e.g., pig) than the recipient. The tissue or cells may be treated with digestive enzymes such as trypsin and collagenase to separate the cells and prepare them for transplantation and/or culturing. Optionally, the cells may be cultured in vitro in order to increase the number of cells for transplant. In certain circumstances, the cells are cultured on a surface coated with gelatin or with poly-L-lysine and laminin in a medium containing the appropriate nutrients and factors for cell growth. The cells may also be altered before being transplanted. In one instance, the genome of the cells may be altered by altering, deleting, or inserting a gene into the genome. The alteration of the genome may be necessary or may aid in the therapeutic effect of the transplant. In another instance, the cells may be treated with certain factors including various nutrients, vitamins, minerals, growth factors, chemical compounds, steroids, hormones, peptides, proteins, or nucleic acids to induce the cells to develop in a certain manner, differentiate, or to de-differentiate. The factors may induce morphologic changes and/or changes in gene expression within the cell. The modified cells are typically more effective in transplantation than the original cell before modification. For example, if myoblasts are transplanted into cardiac myocardium which is diseased because of coronary artery disease, it may be helpful if the transplanted myoblasts secrete or produce an angiogenic factor to induce the development of new capillaries to supply the ischemic area.

If the cells to be transplanted are derived from a donor whose immunological make-up is significantly different from the donor's, the recipient may require immunosuppressive therapy such as steroids and cyclosporin post transplant. The immunosuppressive therapy should substantially suppress rejection of the transplanted cells. The immunosuppressive therapy should best be determined by a medical profession familiar with the transplantation procedure and the recipient's clinical status. In order to avoid the use of immunosuppressive therapy post transplant, the cells to be transplanted may be derived from a related donor, an immediate family, or the recipient himself. As in organ transplants, the closer the HLA match between the donor and the recipient the less likely there will be a rejection of the transplanted cells. To prevent rejection of the transplanted cells, antigens on the surface of the transplanted cells may be modified, masked, or eliminated to prevent or lessen the risk of an immune response from the recipient's immune system (see U.S. Pat. No. 5,283,058, issued Feb. 1, 1994, incorporated herein by reference). In certain embodiments, the MHC class I molecules on transplanted cells are masked with antibodies, antibody fragments (e.g., F(ab′)₂), soluble T-cell receptor fragments, or synthetic organic molecules which mimic the antigen binding properties of T-cell receptors. In certain other embodiments, the cells may be genetically modified to prevent or reduce the risk of T-cell mediated immune response upon transplantation. In certain embodiments, the cells may be derived from a transgenic animal that has been modified to modify or eliminate rejection-inducing surface antigens on the cells of donor tissues. Surface antigens known to interact with host T-cells include MHC class I molecules, LFA-3, and ICAM-1.

The cells once transplanted should preferably respond to the environment in which they are transplanted and thereby integrate themselves and their progeny into the cellular matrix of the tissue/organ which the cells were injected into. The transplanted cells should help to repair an injury to an organ. For example, transplanting skeletal myoblasts into injured myocardium has been shown to increase cardiac output and help repair the site of injury (see U.S. Ser. No. 60/145,849, filed Jul. 23, 1999; U.S. Ser. No. 09/624,885, filed Jul. 24, 2000; and U.S. Ser. No. 10/105,035, filed Mar. 21, 2002, each of which is incorporated herein by reference).

Organs

The agents to be delivered may be injected using the inventive method into any organ, tissue, or tumor within the patient's body. For example, an anti-neoplastic agent may be delivered into a tumor to minimize the effect on surrounding tissues. In certain preferred embodiments, the agents are delivered into an injured site within the organ or tissue. In certain embodiment wherein the agents to be delivered are cells, the cells to be transplanted are injected into an organ at a site which has been injured, is diseased, requires supplementation with additional cells, or requires supplementation with cells with an altered genome. The cells are injected through a needle with a side opening into an organ or tissue under sterile conditions. The delivery of cells may be done during a surgical procedure to minimize the number of tissues and organs the needle must pass through and to better control the delivery of cells. The delivery of cells to a specific site may be guided by various radiological techniques such as fluoroscopy, CT, and x-ray radiology.

The cells are preferably transplanted into a solid tissue or organ rather than a natural hole, lumen, or opening. In certain preferred embodiments, the cells are delivered into an injured, diseased, or damaged site within an organ or tissue. The organs into which the cells may be injected include, for example, cardiac mycocardium, skeletal myocardium, brain, spinal cord, spleen, liver, pancreas, thyroid, adrenal glands, prostate, testes, and ovaries. Without wishing to be bound by a particular theory, the present invention is thought to prevent the leakage or extrusion of the newly injected cells back out of the hole created by a regular needle with the hole at the tip. A closed tip needle is thought to create a hole which better seals itself once the needle is withdrawn, thereby, not allowing the cells, which have just been injected into a closed space, to leak out. This idea becomes increasingly important where the site of injection comes under increasing tension or pressure due to contraction of a muscle, fluid build up inside the organ or tissue, inflammation, cell or tumor growth. etc.

In order to treat a condition, multiple injection may be required at one time or over the course of days, months, weeks, or years. For example, if the transplanted cells are attacked and destroyed by the host's immune system, they will need to be replaced by repeated injections. The course of treatment will be best determined by a professional with experience in treating the patient's condition.

Applications

The inventive method may be applied to the treatment of any disease or condition where the delivery of an agent by injection through a needle into an organ, tissue, or tumor is needed. Just one example of an application of the inventive method is the delivery of cells to a particular organ, tissue, or tumor within a patient's body. Recent research has focused on the use of cellular transplantation in the treatment of various diseases. Researchers have tried to transplant cells into the brains of patients with Parkinson's disease in order to lessen the movement disorders associated with this devastating disease. Researchers have also tried to repair damage to cardiac tissue after myocardial infarction using cellular transplantation. Cellular transplantation into organs which have a limited or no ability to regenerate (e.g., brain, heart) may become increasingly important as researchers learn to control the growth and differentiation of various cell types.

A major problem with the transplantation of adult cardiac myocytes is that they do not proliferate in culture. (Yoon et al. (1995) Tex. Heart Inst. J. 22:119; incorporated herein by reference). To overcome this problem, attention has focused on the possible use of skeletal myoblasts. Skeletal muscle tissue contains satellite cells which are capable of proliferation. Upon purification and expansion of these cells in vitro, they may be injected using a needle with a side opening into the heart at the site of ischemic damage in order to help repair the damaged muscle. The inventive method will allow the transplanted cells to stay at the site of injection so that they can repopulate that area with myocytes and thereby repair the damaged area. Preferably once the cells are injected into the site of injury the cells will repopulate and area and integrate themselves into the already existing network of cells and extracellular matrix.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Bench-top Injections of Human Myoblasts into Pig Hearts

Upon histological examination of post-transplant hearts which had been injected with skeletal myoblasts, it was noticed that the transplanted cells were found at times on the surface of the heart in the epicardial fat rather than at the site of injection. The transplanted cells were thought to have been forced out of the injection site through the needle hole up to the surface of the heart where they began to proliferate. Unfortunately, transplanted cells that are not at the injection site do not provide any aid to the damaged and scarred heart; therefore, various methods of injecting the cells were studied in order to determine the best way to transplant cells into a solid tissue or organ.

Several vials containing frozen human myoblasts were allowed to thaw out on ice. A total of 240×10⁶ cells were washed twice in TX medium. The cells were then split into two tubes. One tube contained 80×10⁶ cells in 1.6 ml of TX medium resulting in a concentration of 50×10⁶ cells/ml. A second tube contained twice as many cells in the same volume of medium resulting in a concentration of 100×10⁶ cells/ml. Cells were then injected into the myocardium of pig heart using various needles, orientations of the needle, sizes of needles, concentrations of cells, volumes injected, and depths of injection. The amount of leakage from the injection site was then measured to determine the best way to inject myoblasts into myocardium and have the cells retained in the tissue at the site of injection.

| | | | | | Depth of | | | | | Time |
|---|---|---|---|---|---|---|---|---|---|---|
| | | | | | Needle | | Orientation | Volume | | before |
| Test | Needle | Gauge | Length | | Insertion | | of Bevel | Injected | Leakage | leakage |
| 1 | Beveled, | 25 G | ⅝ | inch | ⅝ | inch | Up | 100 μl of | 20 μl | 15 sec. |
| | end release | | | | | | | 100 × 10⁶ cells/ml | | |
| 2 | Beveled, | 25 G | ⅝ | inch | ⅝ | inch | Up | 100 μl of | Very | 15 sec. |
| | end release | | | | | | | 100 × 10⁶ cells/ml | little | |
| | | | | | | | | | leakage | |
| 3 | Beveled, | 25 G | ⅝ | inch | ⅝ | inch | Up | 100 μl of | Completely | 15 sec. |
| | end release | | | | | | | 100 × 10⁶ cells/ml | leaked | |
| 4 | Beveled, | 25 G | ⅝ | inch | ⅝ | inch | Down | 100 μl of | 20 μl | 15 sec. |
| | end release | | | | | | | 100 × 10⁶ cells/ml | | |
| 5 | Beveled, | 25 G | ⅝ | inch | ⅝ | inch | Down | 100 μl of | 5 μl | 1 min. |
| | end release | | | | | | | 100 × 10⁶ cells/ml | | |
| 6 | Beveled, | 25 G | ⅝ | inch | ⅝ | inch | Down | 100 μl of | 1 μl | 2 min. |
| | end release | | | | | | | 100 × 10⁶ cells/ml | | |
| 7 | Beveled, | 25 G | ⅝ | inch | ⅝ | inch | Down | 100 μl of | 5 μl | 2 min. |
| | end release | | | | | | | 100 × 10⁶ cells/ml | | |
| 8 | Beveled, | 25 G | 3½ | inches | 1 | inch | | 400 μl of | 1 μl | 1 min. |
| | end release | | | | | | | 100 × 10⁶ cells/ml | leaked | |
| | | | | | | | | | after | |
| | | | | | | | | | some | |
| | | | | | | | | | time | |
| 9 | Beveled, | 25 G | 3½ | inches | 1 | inch | | 400 μl of | 1 μl | 1 min. |
| | end release | | | | | | | 100 × 10⁶ cells/ml | | |
| 10 | Whitacre | 25 G | 3½ | inches | 1 | inch | | 400 μl of | No | 1 min. |
| | side | | | | | | | 50 × 10⁶ cells/ | leakage | |
| | release | | | | | | | ml; wait 30 sec | | |
| | | | | | | | | before | | |
| | | | | | | | | removal | | |
| 11 | Whitacre | 25 G | 3½ | inches | 1 | inch | | 400 μl of | No | 1 min. |
| | side | | | | | | | 50 × 10⁶ cells/ | leakage | |
| | release | | | | | | | ml; wait 30 sec. | | |
| | | | | | | | | before | | |
| | | | | | | | | removal | | |
| 12 | Beveled, | 25 G | 3½ | inches | 1 | inch | ?? | 400 μl of | 10 μl | 1 min. |
| | end release | | | | | | | 50 × 10⁶ cells/ | | |
| | | | | | | | | ml; 30 sec. | | |
| | | | | | | | | wait before | | |
| | | | | | | | | removal | | |
| 13 | Whitacre | 25 G | 3½ | inches | ⅝ | inch | | 100 μl of | 1 μl | 15 sec. |
| | side | | | | | | | 50 × 10⁶ cells/ml | | |
| | release | | | | | | | | | |
| 14 | Whitacre | 25 G | 3½ | inches; | ⅝ | inch | | 100 μl of | 1 μl | 15 sec. |
| | side | | | | | | | 50 × 10⁶ cells/ml | | |
| | release | | ⅝ | inch | | | | | | |

depth

Based on the fourteen injections described in the table above in which two types of release (bevel vs. side release), two cell densities (100×10⁶ vs. 50×10⁶ per ml), two needle lengths (⅝ vs. 3½ inches), and three needles (25 G Whitacre needle 3½ in. side release; 25 G ⅝ in. beveled; 25 G 3½ inches beveled) were tested, the side release needles performed better than the bevel tip needles in preventing leakage. Eight out of nine injections made with the beveled needle leaked whereas with the side release needle two out of four showed no leakage and the other two showed minimal leakage.

A number of factors affect the retention of fluid injected into a tissue. The rate of injection, the total volume injected, the depth to which the needle is inserted into the tissue, and the time delay before removing the needle from the tissue are thought to be some of the more important ones. To compare the relative retention of fluid within the heart tissue after injection with two different needle designs, some of the above listed factors were tested. The table shows that when a beveled needle was used, the needle was inserted to a depth of ⅝ inch, and the cells were injected rapidly (15 sec.) or slowly (1-2 min.), there was always leakage of cells from the injection site (tests #1-7). Although the amount of leakage varied, injecting slower (tests #5-7) seemed to be better than injecting fast (tests #1-4). Increasing the depth of needle penetration appeared to help as well because with an increased needle injection depth to 1 inch (test #8) four times the volume was successfully injected with little difference in leakage compared to that with a ⅝ inch depth injection (test #5). All leakage was stopped by using a side release needle and injecting to a depth of 1 in. over the course of 1 minute (tests #10-11). Returning to a beveled needle resulted in leakage (test #12). To further test the use of a side release needle, 100 μl of fluid was injected rapidly with minimal leakage (tests #13-14), demonstrating the superior retention with the side release needle.

### Example 2

### Autologous Myoblast Transplantation for the Treatment of Ischemic Congestive Heart Failure

Methods: Ischemic Congestive Heart Failure (CHF) was induced in sheep by means of repeated coronary microembolization until the LV ejection fraction (LVEF) was maintained below 35%. Skeletal muscle myoblasts were isolated from biopsies obtained from the front limb of the animal and the cells were cultured until yields of greater than 3×10⁸ cells were achieved. Approximately two weeks after CHF induction, animals were transplanted with autologous skeletal myoblasts (3×10⁸ cells) via direct myocardial injection using a side-port needle. Cells were injected into multiple sites within the ischemically damaged left ventricular wall. Animals were sacrificed 6 weeks after myoblast cell transplantation, the heart was fixed in Formalin, and histological analyses were performed to assess cell survival.

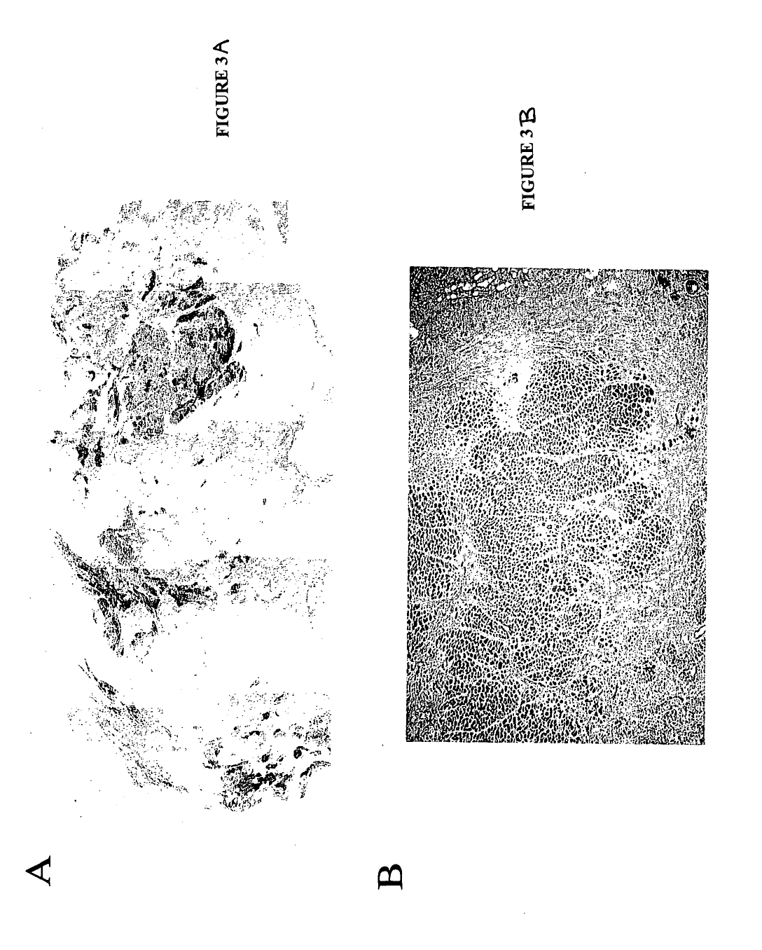

Results: Delivery of cells using a side-port needle resulted in successful cell delivery as assessed by post-mortem histology. The cells, identified by skeletal muscle-specific myosin immunostaining (FIG. 3A), filled large areas of the myocardium into which the cells were injected. On higher magnification, Trichrome stain of one dense cell deposit shows a collection of aligned myotubes (FIG. 3B). The fiber alignment was most often in parallel with the surrounding host myocardium. Each myotube, appearing as small circular red bundles, is a multinucleated fiber cut in cross-section.

Summary: Epicardial injection of autologous myoblasts using a side-port needle successfully delivered cells into ischemically damaged myocardium. Cells remain viable, and fuse to form multinucleated myotubes which most often align with the host myocardium. Histological evidence suggests that the transplanted myoblasts mature and express contractile proteins such as myosin heavy chain.

### Example 3

### Autologous Myoblast Transplantation for the Treatment of Infarcted Myocardium

Purpose of the Protocol. The purpose of this example is to test the feasibility and safety of transplanting autologous myoblasts derived from skeletal muscle into and around the ischemic or scarred areas of the myocardium, post-myocardial infarction. The transplantation of the autologous myoblast cells is performed while the subject is undergoing coronary artery bypass surgery (CABG). The subjects enrolled in the study will have had a myocardial infarction and also have left ventricular dysfunction.

The myoblasts are be expanded in vitro from satellite cells obtained from a biopsy of the subject's skeletal muscle. The cells aree injected into the wall of the left ventricle at the time of the bypass procedure. An objective is to gain preliminary information on the improvement of cardiac function based upon echocardiography and magnetic resonance imaging (MRI) to evaluate regional wall motion, wall thickness, and ventricular volume. The MRI imaging evaluation will be performed in conjunction with other imaging procedures, electrocardiographic measurements and clinical assessments.

Significance and Background for the Study. Coronary artery disease is the leading cause of death in the United States, responsible for 1 of every 4.8 deaths or close to 500,000 deaths each year. The disease is caused by the accumulation of atherosclerotic plaque, consisting of lipid deposits, macrophages, and fibrous tissue, on the walls of vessels supplying heart muscle. According to the American Heart Association, more than 1.8 million Americans have coronary artery disease (AHA, 1999). Rupture of unstable plaques activates substances that promote platelet aggregation and thrombus formation. The thrombus is composed of platelets, blood cells and fibrin that can block one or more of the coronary vessels, resulting in an inadequate supply of oxygen to the heart muscle. This highly active muscle is quickly damaged and the lesions are irreversible because cardiomyocytes, the specialized muscle cells of the heart, do not normally undergo cell division. The end result is an infarct, a damaged area of heart muscle in which necrotic cardiomyocytes are replaced by scar tissue and fibrosis, weakening the contractility and function of the heart. Approximately 1.5 million new and recurring heart attacks are reported each year.

Treatments to prevent ischemic damage after a myocardial infarction include thrombolytic drugs that break down fibrin clots and open up occluded arteries. These drugs have greatly influenced morbidity and mortality from occlusive events, but must be administered within a short interval after a myocardial infarction to be effective. Even with current medical management, about one fifth of acute myocardial infarctions are fatal. Cardiac catheterization, angioplasty, and stenting to open the occluded vessel have proved effective in restoring perfusion but cannot reverse pre-existing ischemic damage. Coronary bypass surgery is often undertaken if none of these procedures is effective. Over 500,000 bypass surgeries are performed annually in the U.S.

The economic consequences associated with cardiovascular disease are staggering as indicated by HCUP Nationwide Inpatient statistics that show that inpatient cost related cardiovascular diseases is the most costly disease category (based on principal diagnosis, 26% or $97 billion) of the total inpatient health care cost. The estimated direct and indirect cost for cardiovascular disease and stroke amounts to $286 billion with $100 billion per year associated with myocardial infarctions.

Those subjects that survive a myocardial infarction and have an area of nonfunctioning myocardium of appreciable size are at increased risk of developing heart failure. The recent advances in therapy do not provide any treatment for myocardial tissue that has been damaged by ischemic events. The left ventricle undergoes enlargement as part of a compensatory mechanism to increase the stroke volume in the weakened muscle. This compensatory response becomes detrimental when the individual cardiomyocyte enlarges resulting in decreased contractility. This remodeling of the myocardium is slowed by pharmacological treatment with ACE (angiotensin-converting enzyme) inhibitors and beta-blockers, but end stage heart failure leads to death in 80% of subjects within five years. Therapies that would replace damaged myocardial tissue and prevent the progression to heart failure would be an important contribution to the treatment of ischemic damage to the myocardium.

If a patient with pre-existing left ventricular dysfunction resulting from previous infarcts requires a CABG procedure, the prognosis becomes less certain. This leaves a need for improved outcome after CABG surgery which in these cases is often combined with ventricular surgical restoration or the placement of an Automatic Intracardiac Cardioverted Defibrillator (AICD). Subjects with impairment at this level are less likely to recover normal cardiac functioning after bypass surgery and might benefit from additional therapy.

While cardiac muscle cells (myocytes) do not have the capacity to divide and repair damaged myocardium, skeletal muscle contains cells, myoblasts, that divide when called upon to repair damaged muscle. Cardiac and skeletal muscle have many similarities in structure, function, and microscopic appearance and thus myoblasts from skeletal muscle may be able to provide contractile function when implanted into the damaged myocardium.

Autologous myoblasts from the subject's skeletal muscle are isolated and expanded to be used for transplantation into damaged heart muscle. Transplantation of myoblasts offers a new treatment that may increase the functional ability of the myocardial wall and decrease ventricular remodeling in the infarct area. Subjects suffering from myocardial infarctions would benefit greatly if these myoblasts could repair their damaged myocardium. These cells are isolated from a muscle biopsy of a subject who has suffered a myocardial infarction and would thus allow transplantation of a subject's own myoblasts into their heart, thereby avoiding any immunological barriers.

The subjects in this study have had a myocardial infarction and have left ventricular dysfunction prior-to undergoing CABG surgery. Left ventricular dysfunction is defined by an ejection fraction below 35%. Patients with left ventricular dysfunction have 90% survival at one month after CABG surgery and 69% survival at five years as compared to 96% and 90% one month and five year survival in patients with normal left ventricular function (ACC/AHA Task Force Report, Circulation 83(3):1125-1173, 1991; incorporated herein by reference). The infarct site is localized prior to the surgery and cells are injected into the infarct and may be injected into surrounding tissue in an attempt to evaluate cell survival and the functional benefit of cell engraftment in the infarct or peri-infarct zones.

Patients for CABG surgery are chosen based on their physical symptoms and assessment of coronary artery occlusion and myocardial perfusion. The most common physical finding is severe angina and if this is combined with evidence of coronary occlusion in a subject who has no contraindications, the procedure is scheduled. The subjects in this study are monitored for cardiac function using MRI to assess regional wall motion, wall thickness, ventricular volume, and ejection fraction prior to surgery. The outcome of CABG surgery for reperfusion of ischemic myocardium is well established. However preexisting left ventricular dysfunction makes the prognosis less certain and scar tissue containing deposits of extracellular matrix is not likely to be affected by re-vascularization and often no attempt is made to graft the scarred tissue.

Preparation of myoblasts for use in humans is performed as described in the art. These myoblasts (satellite cells) reside in skeletal muscle where they act as precursors for myotubes, the muscle fiber cells that have the contractile elements of skeletal muscle. Satellite cells are capable of cell division when the muscle is injured. The myoblasts derived from skeletal muscle can be grown for up to 50 generations in vitro and have been supplied for two clinical trials to treat muscular dystrophy (Neumeyer et al., “Pilot Study of Myoblast Transfer in the Treatment of Becker Muscular Dystrophy” Neurology 51:589-592, 1998; incorporated herein by reference).

A biopsy will be taken from the subject's skeletal muscle and transported to an appropriate laboratory for myoblast isolation and growth. Cells are then harvested and transported back to the clinical site for transplantation.

The use of autologous myoblasts has the advantage that the cells should not be detected as foreign by the immune system. All antigens, presented by antigen presenting cells, will presumably have been seen by the T cells of the host and thus the immune system will be tolerized to these antigens. This was the case in the animal studies performed in support of the study. In these studies, syngeneic myoblasts (Lewis rats) were injected into the heart and did not result in an immune response. In agreement with this finding, use of cyclosporine and steroids in these studies did not confer an advantage to the cells in terms of graft survival.

Animal studies with transplantation of both skeletal myoblasts and fetal cardiac myocytes have demonstrated that skeletal myoblasts can form grafts without compromising the function of the animal's heart (Taylor et al., “Regenerating Functional Myocardium: Improved Performance after Skeletal Myoblast Transplantation” Nature Medicine 4(8):929-933, 1998; Reinecke et al., 1999; each of which is incorporated herein by reference). Myoblasts injected into healthy tissue have been shown to engraft and form myotubes in the heart. Myocardial infarction models have been tested in three laboratories. In rabbits with myocardial infarction, skeletal myoblasts were found to survive, to maintain their skeletal muscle phenotype, and to enhance cardiac function (Taylor et al., “Regenerating Functional Myocardium: Improved Performance after Skeletal Myoblast Transplantation” Nature Medicine 4(8):929-933, 1998; incorporated herein by reference). In a rat cryoinjury model, myoblasts were found to engraft and form myotubes that enhanced cardiac function and became cardiomyocyte-like based upon the expression of heart specific proteins (Murry et al., “Skeletal Myoblast Transplantation for Repair of Myocardial Necrosis” J. Clin. Invest. 98(11):2512-2523, 1996; incorporated herein by reference). Studies have shown that rat myoblasts form stable grafts and enhance myocardial function as measured by a Langendorf procedure (Jain et al., “Skeletal Muscle Implantation Attenuates Post-MI Ventricular Remodeling and Improves Cardiac Performance” 2000; incorporated herein by reference). The cells survived both outside and inside the infarct zone. In addition, the cells fused to form myotubes and appeared to form close contact with the myocytes at the borders of the infarct. Increased myocardial contractility and cardiac output as compared to the control animals may have resulted from actual contraction of the skeletal muscle grafts or from the prevention of increased ventricular volume by the treatment. The end diastolic volume was decreased in the treated animals indicating that the progression of ventricular remodeling observed in the untreated animals had been prevented.

To replace the lost cardiomyocytes in a subject with an infarct of approximately 30% of the left ventricle, it has been calculated that approximately 200 to 300 million new surviving cells are needed. The safety and efficacy of infusing large numbers of myoblasts has been tested in the rat model where one million cells were injected into a 30% infarct. No safety issues were noted in these studies, and rat survival after cell transplantation was excellent.

In summary, myoblast transplantation may be a beneficial therapy for subjects with myocardial infarction and has the potential to repair damaged myocardium. This may be due to the prevention of scarring and expansion of the infarct or by enhanced contractility of the infarcted myocardium resulting from the transplanted myoblasts. Improved regional wall function as measured by MRI can be used to evaluate functional improvement. The imaging can also be utilized for standard cardiac functional assessments such as ejection fraction and cardiac output to determine if the myoblast transplantation leads to increased contractility and prevention of infarct enlargement. Additional imaging, electrocardiographic, and clinical evaluations are also performed to assess cardiac function.

Description of Research Protocol. The purpose of this study is to investigate the feasibility and safety of implanting autologous myoblasts derived from skeletal muscle into the wall of the left ventricle of subjects undergoing CABG surgery following myocardial infarction. Another objective is to obtain preliminary information on graft survival and the effect of the transplant on functional characteristic of the heart.

Study Design. This is a study involving subjects who have experienced a myocardial infarction and have left ventricular dysfunction. The subject is a candidate for CABG surgery and is not a candidate for other surgical procedures, i.e., infarctectomy, ACID, or valvular surgery. Approximately five weeks prior to the surgery, a biopsy is taken from the subject's skeletal muscle. The biopsy is used to generate a population of autologous myoblasts that are implanted at the time of the CABG surgery into a defined area of the heart. The implant region is monitored for its effect on regional wall motion, wall thickness, and ventricular volume.

Subject Selection. Subjects will receive transplants of myoblasts in this study. The number of subjects consented and screened for the study may be larger than eighteen if the biopsy is taken and cells are unable to be sufficiently expanded and harvested before the subject undergoes the CABG surgery or if the subject does not undergo CABG surgery or declines myoblast transplantation at the time of CABG surgery.

The subject's participation consists of baseline procedures, daily visits for 1 week, and then up to 10 visits that will occur within the first two years after cell implantation. If the subject's medical condition necessitates orthotopic heart transplantation (OHT), the myoblast treated heart will be retrieved for testing. Any OHT subjects will be followed through the remainder of the trial period.

Subjects must meet all of the following criteria to be eligible for study participation:

1. Subject must be 18 years of age or older and able to give informed consent.

2. Subject must have a left ventricular ejection fraction of<35% at baseline.

3. Subject must have the approval of his/her cardiologist.

4. Subject must be scheduled for CABG surgery.

5. Subject must have identifiable area of transmural scar within the left ventricle.

6. Subject must be eligible for MRI.

7. Subject must not be a candidate for concurrent ventricular surgical restoration, AICD placement, or valvular surgery.

Subjects who meet any of the following criteria will be ineligible for study participation:

1. Subject has infection that the investigator deems significant to the completion of the procedure.

2. Subject has other complicating cardiovascular abnormalities that the investigator deems significant to the completion of the procedure

3. Subject has clinically significant electrocardiographic abnormalities, e.g.,

High grade atrioventricular block

Frequent, recurrent, or sustained ventricular tachycardia

4. Subject has evidence of skeletal muscle disease.

5. Subject has evidence of other medical conditions that the investigator determines likely to have a significant impact on the outcome of this trial.

6. Subject has active malignancy.

7. Subject has recent history (within past 6 months) of alcohol or drug abuse.

8. If female, subject is pregnant or trying to become pregnant.

Methodology. Subjects are enrolled in the study based upon an initial scheduling for CABG surgery. Following a determination of eligibility for participation in this study, a muscle biopsy is taken from the subject approximately five weeks before the scheduled CABG procedure.

Baseline Evaluations

1. Informed Consent procedures

2. Determination of eligibility criteria

3. History and physical examination

4. Routine blood sampling and laboratory tests to include: hematology (CBC with differential) and blood chemistry including cardiac enzyme levels

5. ECG

6. Echocardiography (per standardized protocol)

7. Urinalysis

8. Blood draw for Diacrin immune testing

9. Muscle biopsy

10. 24 hour Holter monitoring (done two times prior to myoblast transplant)

11. MRI (per standardized protocol)

12. PET scan (optional)

13. NOGA™ mapping (optional)

14. Serum pregnancy test, if female

15. Quality of Life Assessment (optional)

Post Myoblast Transplant Heart Donation

As part of the informed consent the subject is asked to donate his or her heart (treated with autologous myoblasts) for testing if an orthotopic heart transplant should be required after myoblast transplant.

As a part of the informed consent procedure, subjects are asked to consider consenting to an autopsy in the event that the subject dies after receiving myoblasts but prior to OHT. A separate consent form for the autopsy will be made available. See below for details about the histological analysis of the heart.

A muscle biopsy will be taken after a candidate for CABG gives Informed Consent and has met the inclusion/exclusion criteria. The muscle biopsy (approximately 5.0 grams), obtained from the muscle of the arm or leg, taken under sterile conditions, will be transported, using a biopsy transport kit, to a cell processing facility. The facility will inform the investigator two to three days before the cells are ready for transplantation. If the CABG surgery is postponed or cancelled, the cells may be cyropreserved and stored for future implantation. Any unused cells may be used for basic cell transplantation research purposes (e.g., studies on cell growth, storage, freezing, etc.).

Subjects are transplanted with autologous myoblasts derived from skeletal muscle. The myoblast production involves a four-step process. The process involves the procurement of the subject's muscle tissue (biopsy), the receipt and processing of the biopsy to release satellite precursor cells at a cell processing facility, the expansion of myoblasts derived from the satellite cells, and the production of the finished product for transplantation. The production process is performed under the FDA Good Manufacturing Practice regulations (21CFR Part 210) and all applicable FDA guidelines related to cellular/tissue-based therapeutic products.

When a subject scheduled for CABG is enrolled in the trial, cells are expanded and brought to the hospital for transplantation. Isolation of myoblasts will be performed as described herein.

All of the subject's care will be under the supervision of the investigator and sub-investigator(s). Transplantation will occur in the hospital. The subject is prepared for CABG and the procedure will be performed. The intent of this study is to inject cells into an infarcted region of the wall of the left ventricle.

Total cell dose per subject will follow an escalating dose regimen. The first three subjects to undergo the cell transplantation each receive a maximum of 10 million cells. An escalating cell dose of up to 30 million cells is given to the fourth, fifth, and sixth subjects and up to 100 million cells to the seventh, eighth, and ninth subjects. The tenth, eleventh, and twelfth subjects receive the cell dose of up to 300 million cells. The thirteenth, fourteenth, and fifteenth subjects receive the cell dose of up to 600 million cells. The sixteenth, seventeenth, and eighteenth subjects receive the maximum cell dose of up to 900 million cells.

The cells are shipped to the clinical site at a concentration of 0.33×10⁸ cells, 1.0×10⁸ cells per ml or 1.6×10⁸ cells per ml. The concentration of 0.33×10⁸ cells/ml are only used for the lowest dose (10 million cells), and will require 3 injections of 100 μl each to reach the full dose (total volume 300 μl). For the 30 million cell dose group, the cells are concentrated at 1.0×10⁸ cells per ml and will require 3 injections of 100 μl each (total volume 300 μl). For the 100 million cell dose group, the cells are concentrated at 1.0×10⁸ cells per ml and require 10 injections of 100 μl each (total volume 1 ml). For the 300 million cell dose group, the cells are concentrated at 1.0×10⁸ cells per ml and require 30 injections of 100 μl each (total volume 3 ml). For the 600 million cell dose group, the cells are concentrated at 1.6×10⁸ cells per ml and require 25 injections of 150 μl each (total volume 3.75 ml). For the 900 million cell dose group, the cells are concentrated at 1.6×10⁸ cells per ml and require 38 injections of 150 μl each (total volume 5.7 ml). As the target dose increases, the cell concentration is increased to minimize the total volume that is injected.

Each myoblast injection occurs slowly. The needle is kept in place for 5-15 seconds after each injection to minimize cell movement along the injection track. Following the procedure, the subject is transferred from the surigcal suite to the intensive care unit (ICU) for 24-hour observation. Post-surgery, blood (15 ml) is drawn for routine testing.

Study subjects enter the post transplant treatment phase once they receive the autologous myoblasts. The first visit occurs one day after transplant. Subsequent assessment visits occur on days 2 through 6 (or until hospital discharge), weeks 1, 2, 3, 6, 9, 12, and months 6, 9, 12, 18, 24 post-transplantation. The weekly visits (weeks 1, 2, 3, 6, 9, and 12) may be performed within ±3 days of the actual time point. The monthly visits (months 6, 9, 12, 18, and 24) may be performed within ±7 days from the actual time point. During the 24 hours after the transplantation, the subject has continuous standard ICU monitoring of vital signs and clinical condition. Specific potential problems related to the myoblast transplantation procedure include:

1. Arrhythmias

2. Fibrillation during the surgery and injection of the cells

3. Bleeding from injection (implantation) sites in the heart

4. Infection

The safety monitoring is performed by physical exam, ECG, standardized echocardiography, Holter monitoring, blood tests and urinalysis, and Quality of Life assessment (optional). Testing for improved cardiac function is done by standardized MRI and echocardiography, or optionally by PET scan or NOGA™ mapping.

The following evaluations are completed during the Post Transplant Phase.

Day 1

Physical Exam

ECG

Routine Blood Testing

Urinalysis

Adverse Events

Concomitant Medications

Days 2 through 6 (or until hospital discharge)

ECG

Routine Blood Testing

Adverse Events

Concomitant Medications

Week 1

Physical Exam

ECG

Echocardiography

24 hour Holter Monitoring

Routine Blood Testing

Diacrin Blood Testing

Urinalysis

Adverse Events

Concomitant Medications

Week 2

Physical Exam

ECG

Routine Blood Testing

Adverse Events

Concomitant Medications

Week 3

Physical Exam

ECG

Echocardiography

24 hour Holter Monitoring

MRI

PET scan (optional)

Routine Blood Testing

Diacrin Blood Testing

Urinalysis

Adverse Events

Concomitant Medications

Weeks 6 and 9

Physical Exam

ECG

Echocardiography

Routine Blood Testing

Urinalysis

Adverse Events

Concomitant Medications

Week 12

Physical Exam

ECG

Echocardiography

NOGA™ mapping (optional)

MRI

PET scan (optional)

Routine Blood Testing

Diacrin Blood Testing

Urinalysis

Adverse Events

Concomitant Medications

Month 6

Physical Exam

ECG

Echocardiography

24 hour Holter Monitoring

MRI

PET scan (optional)

NOGA™ mapping (optional)

Routine Blood Testing

Diacrin Blood Testing

Urinalysis

Adverse Events

Concomitant Medications

Quality of Life (optional)

Month 9

Physical Exam

ECG

Routine Blood Testing

Adverse Events

Concomitant Medications

Month 12

Physical Exam

ECG

Echocardiography

24 hour Holter Monitoring

MRI

PET scan (optional)

Routine Blood Testing

Urinalysis

Adverse Events

Concomitant Medications

Quality of Life (optional)

Month 18

Physical Exam

ECG

Echocardiography

MRI

PET scan (optional)

Routine Blood Testing

Urinalysis

Adverse Events

Concomitant Medications

Month 24

Physical Exam

ECG

Echocardiography

24 hour Holter Monitoring

Routine Blood Testing

Urinalysis

Adverse Events

Concomitant Medications

Quality of Life (optional)

To summarize, the procedures which study subjects will undergo which are beyond the standard clinical care for subjects with their condition are 1) muscle biopsy 2) implantation of autologous myoblasts; 3) Holter Monitoring, 4) frequent blood draws, and 5) MRI and 6) PET scan and or NOGA mapping (optional, and only if done at baseline), 7) Quality of Life (optional).

Safety evaluations. An adverse event is any undesirable physical, psychological, or behavioral effect experienced by a study subject whether or not the event is considered related to the investigational product. In addition, an adverse event is any unfavorable and unintended sign (i.e., abnormal laboratory finding, symptom, or disease) temporally associated with the use of the investigational product. Symptoms related to a patient's baseline condition or medical history are not reported as adverse events. However, pre-existing conditions that exacerbate during a study are regarded as adverse events. Adverse events will be classified by the body system as suggested by the FDA guidance document “Conducting a Clinical Safety Review of a New Product Application and Preparing a Report on the Review, November 1996.”

A serious adverse event (SAE) is defined as one of the following outcomes: (1) death; (2) life-threatening (any adverse experience that places the subject, in the view of the investigator, at immediate risk of death from the event as it occurred, i.e., does not include a reaction that, had it occurred in more severe form, might have caused death); (3) in-patient hospitalization or prolongation of existing hospitalization; (4) persistent or significant disability/incapacity; (5) important medical event that may jeopardize the subject and may require medical or surgical intervention to prevent one of the other outcomes; and (6) congenital anomaly/birth defect.

An assessment of causality is required for cases of adverse events in clinical investigations. For an adverse event to be associated with the use of the investigational product, there must be a reasonable possibility that the experience may have been caused by the investigational product. Therefore, an associated adverse event implies that there is evidence or argument to suggest a causal relationship between the drug/biologic/treatment and the adverse event.

Starting at the cell transplantation procedure, the investigator will review adverse events and medical intervention will be initiated, if required. Prior to the cell transplantation procedure, only adverse events that are assessed by the investigator as related to the muscle biopsy procedure should be captured and reported.

For all adverse events, the investigator is asked to assess the relationship of the adverse event to the study product, the surgical procedure, and any required treatment. If in accordance with 21 CFR § 312.32, the investigator determines the adverse event to be a serious adverse event, additional steps as described below will be required.

Clinical Assessments. The post-transplantation clinical assessments include: monitoring of adverse events and all concomitant medications, physical examination, ECG, echocardiography (per standardized protocol), 24 hour Holter monitoring, MRI (per standardized protocol), blood testing, urinalysis, PET scan (optional), NOGA™ mapping (optional), Quality of Life assessment (optional), and histological evaluation of heart (if subject undergoes OHT or dies) to assess engraftment.

The investigator performs a physical exam at baseline, day 1, weeks 1, 2, 3, 6, 9, 12, and months 6, 9, 12, 18, and 24. The physical exam includes obtaining blood pressure, heart rate, respiratory rate, temperature, (and at baseline, height and weight), and a documented assessment of the major body systems. Electrocardiograms are used to assess the electrical activity of the heart. ECG are performed at baseline and at all visits post-transplant.

Standardized echocardiography are used to assess cardiac performance, e.g., ventricular systolic and diastolic function. Echocardiography is also used to assess wall thickness. It is done at baseline, weeks 1, 3, 6, 9, 12 and months 6, 12, 18, and 24.

Twenty-four hour Holter monitoring is used to monitor for arrhythmias. Subjects will have this done twice during the baseline period and at weeks 1 and 3, and months 6, 12, and 24.

Standardized MRI is done to assess cell survival and graft function. A MRI is done at baseline, weeks 3 and 12, and months 6, 12, and 18.

Routine blood samples (15 ml) are tested for hematology (including but not limited to: complete blood count with differential) and blood chemistry (including but not limited to: Na, K, CL, CO₂, Glucose, BUN, Creatinine and levels of cardiac enzymes), as a safety assessment at the baseline visit, on the day of transplant, day 1, days 2-6 (or until discharge), weeks 1, 2, 3, 6, 9, 12, and months 6, 9, 12, 18 and 24.

Blood samples (5 ml) are drawn for testing at the baseline, weeks 1, 3, 12, and month 6 visits. Samples are tested for antibodies against the subject's myoblasts. The results from the antibody testing will not affect the clinical care of the subject, but will provide researchers further information on autologous myoblast transplantation.

Routine urinalysis is done as a safety assessment at baseline, day 1, weeks 1, 3, 6, 9, 12, and months 6, 12, 18, and 24.

After a subject who has received a myoblast transplant receives OHT or dies, the portion of the heart that was transplanted with myoblasts is fixed and sectioned for histology. The area containing the transplant is stained with H & E and trichrome to locate the myoblast grafts. The identity of the grafts is confirmed by immunohistochemistry using a myogenin antibody and antibody My32. The size of the graft, cell number, morphology, and extent of infiltration by cells of the immune system is documented.

PET scans are optional tests and can be obtained according to the investigator's discretion at baseline, weeks 3 and 12, and months 6, 12, and 18.

NOGA™ mappings are optional tests and can be obtained according to the investigator's discretion at the baseline, week 12 and month 6.

SF-36 and/or Minnesota Living with Heart Failure are examples of Quality of Life Assessments that may be used at baseline, months 6,12, and 24.

Data Analysis. Safety is assessed with ECG's, blood tests, and physical examinations. Tolerability is assessed by subject reported adverse events.

Risk and Benefit Analysis. While there may be no direct benefits to the subjects who participate in this study, it is hoped that this treatment might reduce signs, symptoms, or other complications associated with infarcted myocardium. The procedure may increase cardiac function and may increase the subject's chance of survival. If autologous myoblasts are successful in this study, others may benefit from this treatment.

The risk factors in this study include possible adverse reactions to the autologous myoblasts. The use of transplanted myoblasts is relatively new and therefore the specific risks are unknown at this time. Animal studies have shown that successful transplantation of muscle cells can be achieved without immunosuppression.

There is a risk of infection and other complications related to the removal of the muscle tissue for the preparation of cells for the transplantation. If an infection does occur, it can be treated. There is also a risk of bleeding, bruising, or hematoma at the biopsy site. Biospy is obtained using a local anesthetic, but pain from the procedure may be experienced. There is a slight risk that scarring may occur at the biopsy site.

Because this is an autologous, cellular-based product, allergic reactions are possible but of low probability. The subject should be closely monitored during the perioperative period for any allergic reaction and if a reaction does occur it should be treated per clinical standards of care.

There is a risk of bleeding and/or clot development at the injection sites in the left ventricle. The subject should be closely monitored and clinical standards of care should be employed to manage bleeding and/or clot development. There is a possibility that the occurrence of this risk may lead to death or disability.

The risk of infection from the autologous myoblast transplantation at the transplant site is of low probability. The subject should be closely monitored during the perioperative period for any signs and symptoms of infection and if an infection does occur, it should be treated per clinical standards of care. There is a possibility that the occurrence of infection may lead to death or disability.

There is a risk of arrhythmias associated with the engraftment of skeletal muscle in the heart. Arrhythmias were not found to occur in the pre-clinical studies. Undetected and untreated arrhythmias may cause death. Subjects are monitored closely for the development of a heart arrhythmia.

Injecting myoblasts in the wall of the left ventricle may cause a decrease in left ventricular functioning. The risk of this occurrence is unknown but may lead to left heart failure and/or death.

The risk of injury as a result of intracardiac mapping is very low and is similar to standard percutaneous cardiac intervention procedures.

There is a risk that the transplanted cells will be rejected by the subject or fail due to causes other than immune rejection. Effects of rejection may include rash, fever or hypertension. It is unknown what effect, if any, graft failure may have on the subject's medical condition.

It is not known how long myoblasts will survive in the human heart. Animal studies and human experience to date indicate grafts integrated properly in the heart survive for at least three months.

### Example 4

### Autologous Myoblast Transplantation for the Treatment of End-stage Heart Failure

Purpose of the Protocol. The purpose of this Example is to test the feasibility and safety of transplanting autologous myoblasts derived from skeletal muscle into the myocardium of subjects in end stage heart failure. The subjects are candidates for heart transplant surgery and are scheduled for placement of a left ventricular assist device (LVAD) as a bridge to orthotopic transplantation. The cells are prepared from tissue obtained from a biopsy of the subject's skeletal muscle and are transplanted into the subject's heart in a defined region of the left ventricle. The cells are injected directly into the myocardium during the surgery to implant the LVAD. The myoblasts are expanded in vitro from the satellite cells obtained from the biopsy. Safety is evaluated based upon any unexpected adverse events, such as abnormal cardiac function, that might be due to the implantation of the myoblasts. A secondary objective is to gain preliminary information on the autologous graft survival and the potential for improvement of cardiac function that might be associated with the autologous myoblast transplantation.

Significance and Background for the Study. Heart failure is the cause of more than one million hospitalizations per year and is the most common hospital diagnosis in patients over age 65. Approximately 70,000 people with heart failure could benefit from cardiac replacement each year, but only about 2,500 heart transplants are performed (Hosenpub et al. “The Registry of the International Society for Heart and Lung Transplantation” J. Heart Lung Transplant. 16:691-712, 1997; incorporated herein by reference). Heart failure is the major cause of death from cardiovascular disease. One in five patients with a diagnosis of heart failure will die within one year and 50% of patients will be dead within 5 years. The economic consequences associated with cardiovascular diseases are staggering as indicated by HCUP (Healthcare Cost and Utilization Project) nationwide inpatient statistics showing that cardiovascular diseases are the most costly disease category, representing 26% of the total inpatient health care cost based on principal diagnosis or $97 billion. Management of heart failure in hospitals costs about $36 billion annually (O'Connell et al. “Economic impact of heart failure in the United States: time for a different approach” J. Heart Lung Transplant 13: S107-S112, 1995; incorporated herein by reference). Therapies that would replace damaged myocardial tissue and prevent the progression of heart failure would be an important contribution to the treatment of this disease.

Heart failure is the end stage of a destructive cycle initiated by an underlying cardiovascular disease resulting in a pathologic stress to the heart which in turn leads to a compensatory mechanism that precipitates further damage to the heart. These changes to the heart wall can be classified as to whether they affect systolic or diastolic function of the heart. Thus, heart failure may be ultimately due to an inadequacy of the pumping action (systolic heart failure), a defect in ventricular filling (diastolic heart failure), or a combination of both deficiencies.

The major risk factors for heart failure are hypertension, which increases risk by 200 percent, diabetes, coronary artery disease, previous myocardial infarction, infections, and valve defects. A single risk factor can cause heart failure, but combinations of factors significantly increase the risk. Patients can present with dyspnea, fatigue, and edema of the feet, ankles, and legs. Excess fluid in the lungs can also cause persistent coughing or wheezing. A patient history to assess risk factors and physical exam to detect the above symptoms as well as abnormal heart sounds and lung congestion can provide a diagnosis of heart failure. Further confirmation of heart failure is available from ECG, echocardiography, and chest X-ray.

Diastolic heart failure results from an abnormality in ventricular filling, which may be due to the ventricle's reduced compliance caused by replacement of distensible tissue with fibrotic non-distensible scar tissue. In this condition the diastolic volume is slightly less than normal but the left ventricular pressure is increased throughout diastole. The elevation of ventricular pressure results in high upstream venous pressure causing pulmonary and systemic congestion. Contractile performance, i.e., stroke volume and ejection fraction, is normal or near normal. Compensatory dilation of the left ventricle leads to increased diastolic volume and consequently elevated diastolic pressure. Importantly, decreased contractility leads to lower stroke volume and ejection fraction.

Coronary artery disease results in both diastolic and systolic dysfunction. Inadequate supply of oxygen to this highly active muscle causes rapid damage and lesions that are thought to be irreversible. The end result is an infarct: a damaged area of heart muscle in which necrotic cardiomyocytes are replaced by scar tissue and fibrosis. In this condition, systolic failure is due to chronic loss of contractility from the loss of cardiomyocytes. Diastolic failure is caused by increased chamber stiffiess due to the incursion of nondistensible scar tissue.

The type of stress placed upon the ventricular wall determines the physiology of heart failure. Hypertension or aortic stenosis stresses the myocardium due to pressure overload. Pressure overload leads to systolic wall stress during left ventricle ejection since the heart must contract with a greater than normal force to pump an adequate volume of blood. The increased afterload causes left ventricle dilation, which in turn leads to the myocardial hypertrophy. Aortic regurgitation, a back flow of blood through a “leaky” aortic semilunar valve, results in volume-overload as the left ventricle receives blood during both systole and diastole. Over time, this causes a dilation of the left ventricle. The increased diameter of the dilated ventricle in turn leads to an increased preload as the sarcomeres of the myofibrils are extended beyond their normal maximal length.

The primary mechanism to compensate for the increased load (pressure or volume) is ventricular hypertrophy. Since cardiomyocytes cannot divide, myocardial hypertrophy results from an increase in the size of individual myocytes without an increase in the number of cells. The pattern of hypertrophy differs depending upon whether the stress is related to volume or pressure overload. Thus, although the mass of the left ventricle increases to the same extent as a result of pressure or volume overload, the wall thickness increases more in pressure overload than in volume overload. At the cellular level, pressure overload leads to myocardial hypertrophy that develops in a concentric fashion resulting from parallel replication of myofibrils and thickening of individual myocytes. Volume overload and diastolic wall stress lead to replication of sarcomeres in series, elongation of myocytes, and ventricular dilation. The extent of ventricular enlargement that results from volume overload is greater than that from pressure overload. In both conditions, the primary result of the hypertrophic compensatory response is to return systolic wall stress to normal levels and improve cardiac output. However, under the continued hemodynamic stress, further cellular changes occur, creating a downward spiral. These changes include a breakdown of myofibrils and the tubular system, a displacement of cardiac tissue with fibrotic tissue and myocyte necrosis. Apoptosis of cardiomyocytes plays an important role in the loss of contractile tissue. This causes increased stress on the remaining cardiomyocytes leading to reactive hypertrophy and heart failure. In addition to these changes to the cardiomyocytes, hypertrophy may also cause a decrease in capillary density with resulting ischemia, which will quicken replacement fibrosis.

Patients who are identified for placement of an LVAD are in end stage heart failure and have few therapeutic options other than heart transplantation or installation of a cardiac assist device. The patients who progress to this point have generally been treated by pharmacological therapy but have ongoing ventricular remodeling that has become life threatening in the absence of a transplant. Treatment of patients with early stage heart failure is initiated with ACE inhibitors, diuretics, inotropic agents, and β-blockers. The use of ACE inhibitors has been shown to slow the progression of left ventricular remodeling, but the process is not halted by any pharmacological therapy.

The use of LVADs has greatly increased survival of patients who are to receive an orthotopic heart transplant. Clinical trials with the HeartMate System (ThermoCardiosystems, Inc.) have demonstrated a 55 percent reduction in mortality in heart transplantation candidates (Frazier et al. “The HeartMate Left Ventricular Assist System: overview and 12-year experience” Tex. Heart Inst. J. 25:265-271, 1998; incorporated herein by reference). The number of transplant candidates who received a transplant was 70 percent when supported with the BVS 500 (Abiomed, Inc.) and support was provided for up to 98 days. An average of 87 percent of patients supported on LVADs survive to hospital discharge after heart transplantation (Burton et al. Ann. Thorac. Surg. 55:1425-1430, 1993; incorporated herein by reference).

While cardiac myocytes do not have the capacity to divide and repair damaged myocardium, skeletal muscle contains cells called satellite cells that divide as myoblasts when called upon to repair damaged muscle. However, both types of striated muscle, i.e., cardiac muscle and skeletal muscle, have many similarities in structure, function, and microscopic appearance; and thus myoblast from skeletal muscles may be able to provide contractile function when implanted into the damaged myocardium (Murry et al. “Skeletal Myoblast Transplantation for Repair of Myocardial Necrosis” J. Clin. Invest. 98(11):2512-2523 (1996); Taylor et al. “Regenerating functional myocardium: Improved performance after skeletal myoblast transplantation” Nature Medicine 4(8):929-933, 1998; each of which is incorporated herein by reference). Autologous myoblasts from the subject's skeletal muscle are isolated, expanded, and used for transplantation into damaged heart muscle. Transplantation of myoblasts offers a new treatment that may increase the functional ability by replacing dead myocytes within the necrotic myocardial wall. The transplanted myoblasts are intended to help to re-establish a normal systolic wall stress and end or retard the destructive cycle of heart failure. Thus, by relieving wall stress by replacement therapy, the detrimental hypertrophic changes will no longer continue to occur and the cells that are hypertrophic may have the opportunity to return to a normal morphology. While it may appear paradoxical, the addition of myoblasts may actually reduce the size of the heart and thereby prevent heart failure. Cells will be isolated from a muscle biopsy of a subject who is suffering from congestive heart failure and would thus allow transplantation of a subject's own myoblasts into their heart, thereby avoiding any immunological barriers.

The subjects have been diagnosed with heart failure. The subjects will have autologous myoblasts implanted directly into the heart muscle during the LVAD implantation surgery. The myoblast transplantation is intended to repopulate the heart muscle with contractile myocytes that might reduce the myocardial dilation of the heart and thereby improve its function. In addition, when the subject goes on to receive a heart transplant, the heart that is removed is used for histological analysis to assess myoblast survival.

Preparation of myoblasts for use in humans has been described herein. These myoblasts derived from satellite cells reside in skeletal muscle where they act as precursors for myotubes, the muscle fiber cells that have the contractile elements of skeletal muscle. The skeletal satellite cells are capable of cell division when the muscle is injured and thereby replace the injured muscle. The myoblasts derived from skeletal cells can be grown for as many as 50 generations in vitro and have been supplied for the two clinical trials mentioned above to treat muscular dystrophy.

For this study a biopsy are taken from the subject's skeletal muscle and transported to a cell processing facility for myoblast isolation and growth. The myoblasts are grown for 3-5 weeks, harvested, and the finished product transported to the clinical site. Alternatively, the muscle tissue and/or the expanded myoblast may be cryopreserved for future transplantation. The myoblasts are then implanted directly into the myocardium of the subject's left ventricle at the time of the LVAD surgery.

The use of autologous myoblasts has the advantage that the cells will not be detected as foreign by the immune system. All antigens present in the autologous graft presumably have been encountered by the immune system of the host and thus the recipient is tolerant to these antigens. The animal studies using syngeneic donor rats that were performed in support of this clinical study mimics the use of autologous myoblast. The syngeneic myoblasts (Lewis rats) were implanted into the heart and there was no significant immune response to the graft.

Animal studies with transplantation of both skeletal and cardiac myocytes have demonstrated that skeletal myoblast cell lines as well as cultured myoblasts isolated from skeletal muscle can form grafts without compromising the function of the animal's heart. Myoblasts injected into healthy tissue have been shown to engraft and form myotubes in the heart. Myocardial infarction models have been tested in three laboratories. In rabbits with cryoinjury, skeletal myoblasts were found to survive, to maintain their skeletal muscle phenotype, and to enhance cardiac function. In a rat cryoinjury model, myoblasts were found to engraft and form myotubes that again enhanced cardiac function and became cardiomyocyte-like based upon the expression of heart specific proteins. Studies performed by Jain et al. (“Cell Therapy Attenuates Deleterious Ventricular Remodeling and Improves Cardiac Performance After Myocardial Infarction” Circulation 103:1920-1927, 2001; incorporated herein by reference) have shown that rat myoblasts form stable grafts and enhance myocardial function as measured by a Langendorff procedure as well as by treadmill capacity. The end diastolic volume was decreased in the treated animals indicating that the implanted myoblast had prevented the progression of ventricular remodeling observed in the untreated animals. The cells survived both outside and inside the infarct zone. In addition, the cells fused to form myotubes and appeared to form close contacts with the myocytes at the borders of the infarct. Increased myocardial contractility and cardiac output as compared to the control animals may have resulted from actual contraction of the skeletal muscle grafts or by the prevention of increased ventricular volume (dilation) by the treatment.

To repopulate the left ventricle with autologous myoblasts to achieve a 30% increase in contractile cells to replace the destroyed cardiomyocytes, the implantation of at least 300 million cells is required. The safety and efficacy of implanting large numbers of myoblasts has been tested in the rat model where one million cells were injected into 30% of the left ventricle. Safety issues have not been noted in these studies, and rat survival after cell transplantation has been excellent.

In summary, myoblast transplantation may be a beneficial therapy for subjects with heart failure by repopulating heart muscle that has a loss of contractile myocytes. This may prevent the progression of heart failure by relieving the stress that contributes to the destructive cycle of myocardial breakdown. Histopathological analysis of tissue obtained at the time of OHT will allow examination for the survival of transplanted myoblasts. By using autologous myoblasts in these pilot studies, immunological issues usually associated with transplantation will be avoided.

Description of Research Protocol. The purpose of this study is to investigate the feasibility and safety of implanting autologous myoblasts derived from skeletal muscle into the myocardium of subjects with left heart failure undergoing LVAD surgery as a bridge to orthotopic heart transplant.

Another objective is to obtain preliminary information on graft survival and the effect of the transplant on functional characteristics of the heart.

This is an open study involving subjects who are in end stage heart failure. The subjects are candidates for LVAD surgery as a bridge to orthotopic heart transplant. When the investigator has determined that the subject is expected to require LVAD surgery, a sterile biopsy is taken from the subject's skeletal muscle. The biopsy is used to generate a population of autologous myoblasts that are implanted at the time of the LVAD surgery directly into a defined area of the hypertrophic heart.

The subject's participation consists of baseline procedures, daily visits for 1 week, and then up to 15 visits that will occur within the first two years after cell implantation or until orthotopic heart transplantation.

Subjects must meet all of the following criteria to be eligible for study participation:

1. Subject must be 18 years of age or older and able to give informed consent

2. Subject has been determined to be a candidate for LVAD implantation

3. Subject is a candidate for orthotopic heart transplantation

4. Subject consents to donate their heart (treated with autologous myoblasts) for testing at the time of orthotopic heart transplant

Subjects who meet any of the following criteria will be ineligible for study participation:

1. Subject has sepsis, pneumonia, and other active infections (by urine, blood cultures, or chest X-ray) at the time of cellular transplantation.

2. Subject has other complicating cardiovascular abnormalities that the Investigator deems significant to the completion of the procedure

3. Subject has evidence of skeletal muscle disease

4. Subject has evidence of other medical conditions that the Investigator determines likely to have a significant impact on the outcome of this trial.

5. Subject has active malignancy

6. Subject has recent history (6 months) of alcohol or drug abuse.

7. If female, pregnant or trying to become pregnant

Subjects are enrolled in the study based upon an initial determination of a need for LVAD surgery. Following a determination of eligibility for participation in this study, a muscle biopsy is taken from the subject and all other baseline evaluations are completed as close as possible to the anticipated LVAD procedure.

Baseline Evaluations

1. Informed Consent procedures

2. Determination of eligibility criteria

3. History and physical examination

4. Routine blood sampling and laboratory tests to include: hematology (CBC with differential) and blood chemistry including cardiac enzyme levels

5. ECG

6. Echocardiography

7. Urinalysis

8. Blood draw for Diacrin immune testing

9. Muscle Biopsy

10. Serum pregnancy test for women of child bearing potential

As part of the informed consent the subject agrees to donate their heart (treated with autologous myoblasts) for testing at the time of orthotopic heart transplant.

As a part of the informed consent procedure, subjects are asked to consider consenting to or allowing for an autopsy in the event that the subject dies after receiving myoblasts but prior to OHT. See below for details about the histological analysis of the heart.

A muscle biopsy is taken after a candidate for LVAD surgery gives Informed Consent and has met the inclusion/exclusion criteria. The muscle biopsy (up to 1.0-5.0 grams), taken under sterile conditions, is transported to a cell processing facility where myoblasts will be isolated and expanded in culture. The facility will inform the investigator two to three days before the cells are ready for infusion. The investigator and facility coordinate scheduling of the harvest of the autologous myoblasts, and the cells are transported to the hospital for the treatment. In the event that the LVAD surgery is postponed and the desired myoblast cells dosage has been attained, cells can be frozen and stored for a future transplantation. At the time the cells are needed, they are thawed and transported to the clinical facility.

If the subject dies, undergoes OHT prior to LVAD implantation and cell treatment, requires the LVAD before the cells are ready for transplantation, or does not undergo the LVAD surgery, the cells may be used for basic cell transplantation research purposes.

Subjects will be transplanted with autologous myoblasts derived from skeletal muscle. The myoblast production involves a four step process that involves the procurement of the subject's muscle tissue (biopsy), the receipt and processing of the biopsy to release satellite precursor cells, the expansion of myoblasts derived from the satellite cells, and the production of the finished product for transplantation. The production process is performed under the FDA Good Manufacturing Practice regulations (21CFR Part 210) and all applicable FDA guidelines related to cellular/tissue-based therapeutic products.

When a subject who is a potential candidate for a LVAD is enrolled in the study, autologous myoblasts will be expanded and brought to the hospital for infusion at the time of LVAD surgery. The subject is identified based on the inclusion/exclusion criteria for the trial. Isolation of myoblasts is performed as described above.

On the day of the procedure, prior to myoblast transplantation, blood is drawn for routine testing (15 ml). All of the subject's care will be under the supervision of the investigator and sub-investigator(s). Transplantation will occur in the hospital. The subject is prepared for the LVAD and the procedure is performed.

The cells are at a concentration of 8×10⁷ cells per ml. Injections of up to 100-500 μl will be made into up to 30 sites in the infarct and peri-infarct zones of the left ventricle. A maximum of 300×10⁶ cells is injected. The needle is kept in place for at least 30 seconds after each injection to minimize cell movement along the injection track. Following the procedure, the subject is transferred from the surgical suite to the intensive care unit for 24-hour observation.

Study subjects enter the post transplant treatment phase once they receive the autologous myoblasts. The first visit occurs one day after transplant. Subsequent assessment visits occur on Days 2 through 6, Week 1, Days 9 and 11, Weeks 2, 3 and 4, Months 2, 3, 4, 5, 6, 9, 12, 18, and 24 post-transplantation. The Week 3 and 4 visits are performed within ±3 days of the actual time point. The monthly visits (Months 2, 3, 4, 5, 6, 9, 12, 18, and 24) are performed within ±7 days from the actual time point. The treatment phase ends and the assessment schedule terminates at the time of orthotopic heart transplantation (OHT). If orthotopic heart transplantation occurs after 24 months, arrangements is made through the subject's primary care physician to track the subject to insure retrieval of the myoblast treated heart for testing at the time of OHT or death. In addition to routine clinical testing of blood at the site, blood is collected for periodic testing. During the 24 hours after the transplantation, the subject has continuous standard ICU monitoring of vital signs and clinical condition. Specific potential problems related to the myoblast transplantation procedure include:

1. Arrhythmias

2. Fibrillation during the surgery and injection of the cells

3. Bleeding from injection (implantation) sites in the heart

4. Infection

The following evaluations will be completed during the treatment phase:

Day 1

Physical Exam

ECG

Routine Blood Testing

Urinalysis

Adverse Events

Concomitant Medications

Days 2 through 6

ECG

Adverse Events

Concomitant Medications

Week 1

Physical Exam

ECG

24 hour Holter Monitoring

Routine Blood Testing

Diacrin Blood Testing

Urinalysis

Adverse Events

Concomitant Medications

Days 9 and 11

ECG

Adverse Events

Concomitant Medications

Weeks 2 and 3

Physical Exam

ECG

Adverse Events

Concomitant Medications

Week 4

Physical Exam

ECG

Echocardiography

24 hour Holter Monitoring

Routine Blood Testing

Diacrin Blood Testing

Urinalysis

Adverse Events

Concomitant Medications

Months 2, 4, 5 and 9

Physical Exam

ECG

Routine Blood Testing

Adverse Events

Concomitant Medications

Month 3

Physical Exam

ECG

Routine Blood Testing

Diacrin Blood Testing

Urinalysis

Adverse Events

Concomitant Medications

Month 6

Physical Exam

ECG

Echocardiography

24 hour Holter Monitoring

Routine Blood Testing

Diacrin Blood Testing

Urinalysis

Adverse Events

Concomitant Medications

Month 18

Physical Exam

ECG

Routine Blood Testing

Urinalysis

Adverse Events

Concomitant Medications

Months 12 and 24

Physical Exam

ECG

Echocardiography

24 hour Holter Monitoring

Routine Blood Testing

Urinalysis

Adverse Events

Concomitant Medications

To summarize, the procedures which study subjects undergo which are beyond the standard clinical care for subjects with their condition are 1) implantation of autologous myoblasts; 2) Holter Monitoring and 3) frequent blood draws.

Safety evaluations. An adverse event is any undesirable physical, psychological, or behavioral effect experienced by a study subject whether or not the event is considered related to the investigational product. In addition, an adverse event is any unfavorable and unintended sign (i.e., abnormal laboratory finding, symptom, or disease) temporally associated with the use of the investigational product. Symptoms related to a patient's baseline condition or medical history are not reported as adverse events. However, pre-existing conditions that exacerbate during a study are regarded as adverse events. Adverse events will be classified by the body system as suggested by the FDA guidance document “Conducting a Clinical Safety Review of a New Product Application and Preparing a Report on the Review, November 1996.”

A serious adverse event (SAE) is one resulting in one of the following outcomes: (1) death; (2) life-threatening (any adverse experience that places the subject, in the view of the investigator, at immediate risk of death from the reaction as it occurred, i.e., does not include a reaction that, had it occurred in more severe form, might have caused death); (3) in-patient hospitalization or prolongation of existing hospitalization; (4) persistent or significant disability/incapacity that requires or prolongs hospitalization; (5) important medical event that may jeopardize the subject and may require medical or surgical intervention to prevent one of the other outcomes; and (6) congenital anomaly/birth defect.

Clinical Assessments. An important aspect of the post-transplantation clinical assessment is to closely monitor adverse events, physical examination, ECG, echocardiography, 24 hour Holter monitoring, MRI and histological evaluation of heart (after subject undergoes OHT or dies) to assess engraftment. These assessments will be performed during defined scheduled visits.

The investigator will perform a physical exam at Baseline, Day 1, Weeks1, 2, 3, 4 and Months 2, 3, 4, 5, 6, 9, 12, 18, and 24. The physical exam includes obtaining blood pressure, heart rate, respiratory rate, temperature, height, weight, and a documented assessment of the major body systems.

Electrocardiograms will be use to assess the electrical activity of the heart. ECG will be performed at Baseline, Day 1, Days 2-6, Week 1, Days 9 and 11, Week 4 and Months 2, 3, 4, 5, 6, 9, 12, 18, and 24.

Echocardiography will be used to assess cardiac performance, e.g., ventricular systolic and diastolic function. Echocardiography is also used to assess wall thickness. It is done at Baseline, Week 4 and Months 6, 12 and 24.

Twenty-four hour Holter monitoring is done to monitor for arrhythmias. Subjects will have this done at Week 1, Week 4 and Months 6, 12, and 24.

Routine blood samples (15 ml) are tested for hematology (including but not limited to: completed blood count with differential) and blood chemistry (including but not limited to: Na, K, Cl, CO₂. Glu, BUN, Creat) including levels of cardiac enzymes, as a safety assessment at the Baseline visit, on the Day of Transplant, Day 1, Week 1, Week 4 and Months 2, 3, 4, 5, 6, 9, 12, 18 and 24.

Blood samples (5 ml) are drawn for testing at the Baseline, Week 1, Week 4 and Months 3 and 6 visits. Samples will be tested for antibodies against the subject's myoblasts. The results from the antibody testing will not affect the clinical care of the subject, but will provide researchers further information on autologous myoblast transplantation.

Routine urinalysis is done as a safety assessment at Baseline, Day 1, Week 1, Week 4, and Months 3, 6, 12, 18, and 24.

After a subject who has received a myoblast transplant receives OHT or dies, the heart that was transplanted with myoblasts is fixed and sectioned for histology. The area containing the transplant are stained with H & E and trichrome to locate the myoblast grafts. The identity of the grafts are confirmed by immunohistochemistry using a myogenin antibody and antibody My32. The size of the graft, cell number, morphology, and extent of infiltration by cells of the immune system are documented.

Data Analysis. Safety will be assessed with blood tests and physical examinations. Tolerability is assessed by subject reported adverse events. The purpose of this investigation is to determine the safety of implantation of autologous myoblasts into the heart of subjects with ischemic or scarred myocardium.

Risk and Benefit Analysis. Although most heart failure subjects that undergo LVAD surgery go on to receive OHT, a significant percentage does not survive to transplant. While there may be no direct benefits to the subjects who participate in this trial, it is hoped that this treatment might reduce signs, symptoms, or other complications associated with heart failure. The procedure may stabilize the heart and slow the progression of ventricular remodeling and may increase the subject's chance of survival to transplant.

The risk factors in this study include possible adverse reactions to the autologous myoblasts. The use of transplanted myoblasts is relatively new and therefore the specific risks are unknown at this time. Animal studies have shown that successful transplantation of muscle cells can be achieved without immunosuppression.

There may be unknown risks associated with transplantation of autologous myoblast cells.

All participating investigators will be notified if any new risks as they are identified.

At this time it is not known how long myoblasts will survive in the human heart. Animal studies indicate grafts integrated properly in the heart survive for as long as the experiment was run (three months).

### Example 5

### Survival of Autologous Myoblasts Transplated into Infarcted Human Myocardium

This example describes a study in which autologous skeletal myoblasts were isolated from a human subject, processed and expanded in tissue culture, and then delivered to the patient's heart using a Whitacre pencil point needle with a side opening while the patient was undergoing implantation of a left ventricular assist device (LVAD) while awaiting heart transplantation. The Clinical Phase I study was approved by the Institutional Review Board for Human Studies (LGH-Bryant Heart Ctr.) and was conducted in accordance with federal guidelines under an approved IND and informed consent process. The patient died 5 days after surgery and the patient's heart was retrieved, and analyzed.

Study Subject and Protocol: The patient was evaluated and approved for heart transplantation and underwent study recruitment and muscle biopsy. The muscle biopsy was taken from the right quadriceps muscle under sterile conditions using local anesthetics. The muscle specimen was immediately placed in transport medium and sent to the GMP isolation facility.

The patient was evaluated and underwent HeartMate® LVAD (Thoratec, Inc.) implantation as a bridge to heart transplantation. At the time of LVAD implantation, multiple injections of autologous skeletal myoblasts were made into the anterior wall of the left ventricle using a 3.0 inch long 26 gauge Whitacre pencil point needle with a side opening. Injection location was selected based upon echocardiography prior to surgery, and direct visualization during the open heart surgery. Fifteen 100 μL injections were delivered at a constant slow rate of delivery. An additional fifteen 100 μL injections were delivered approximately 1 cm apart with a one-inch long 26-gauge needle. The needle was kept in place for at least 30 seconds after each injection to minimize cell movement along the injection track. All of the injections were made into a designated area of approximately 3×3 cm² demarcated with surgical clips. The LVAD implant procedure was completed in the usual fashion. The patient died 5 days after surgery and his heart was retrieved for analysis.

Preparation of Autologous Skeletal Myoblasts: The starting10 grams of skeletal muscle obtained at biopsy was stripped of connective tissue, minced into a slurry in digestion medium, and then subjected to several cycles of enzymatic digestion at 37° C. with 1× trypsin/EDTA (0.5 mg/mL trypsin, 0.53 mM EDTA; GibcoBRL) and collagenase-hepatocyte qualified (0.5 mg/mL; GibcoBRL) to release satellite cells. Skeletal myoblast cultures were expanded according to a modified Ham's method (see Ham, R. G., et al., Adv. Exp. Med. Biol. (1990), 280:193-9, the entire teachings of which are incorporated herein by reference). Satellite cells were plated and grown in myoblast basal growth medium (SkBM; Clonetics) containing 15-20% fetal bovine serum (Hyclone), recombinant human epidermal growth factor (rhEGF: 10 ng/mL), and dexamethasone (3 μg/μL). The cells were grown for 11-13 doublings to achieve the final yield of 300 million cells. To avoid any possibility of myotube formation during the culture process, cell densities were maintained throughout the process so that<75% of the culture surface was occupied by cells.

Prior to transplantation, in excess of 300 million cells were washed and suspended in transplantation medium at approximately 100 million cells per cc and loaded into five 1 cc tuberculin syringes. The cells were kept at 4° C. during transport. Sterility tests were conducted on the final product as well as throughout the digestion and expansion procedures.

Histological Analysis and Immunohistochemical Techniques: Excised myocardium was fixed in formalin, cut into small blocks, and paraffin embedded. Six micron thick sections were cut, mounted, and stained with trichrome.

Results

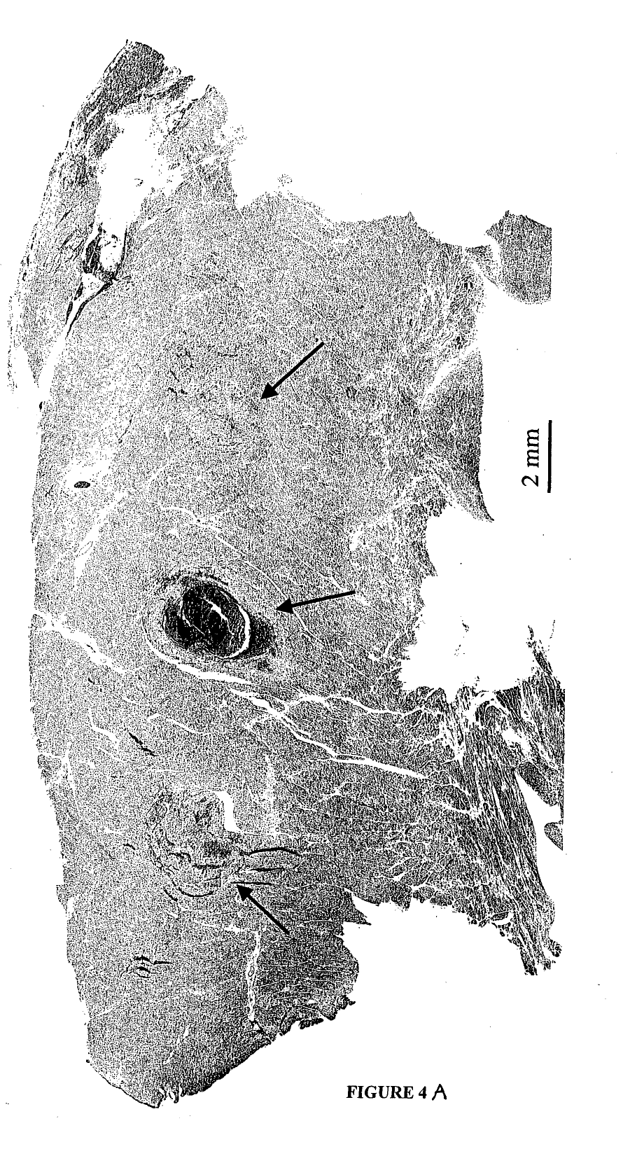

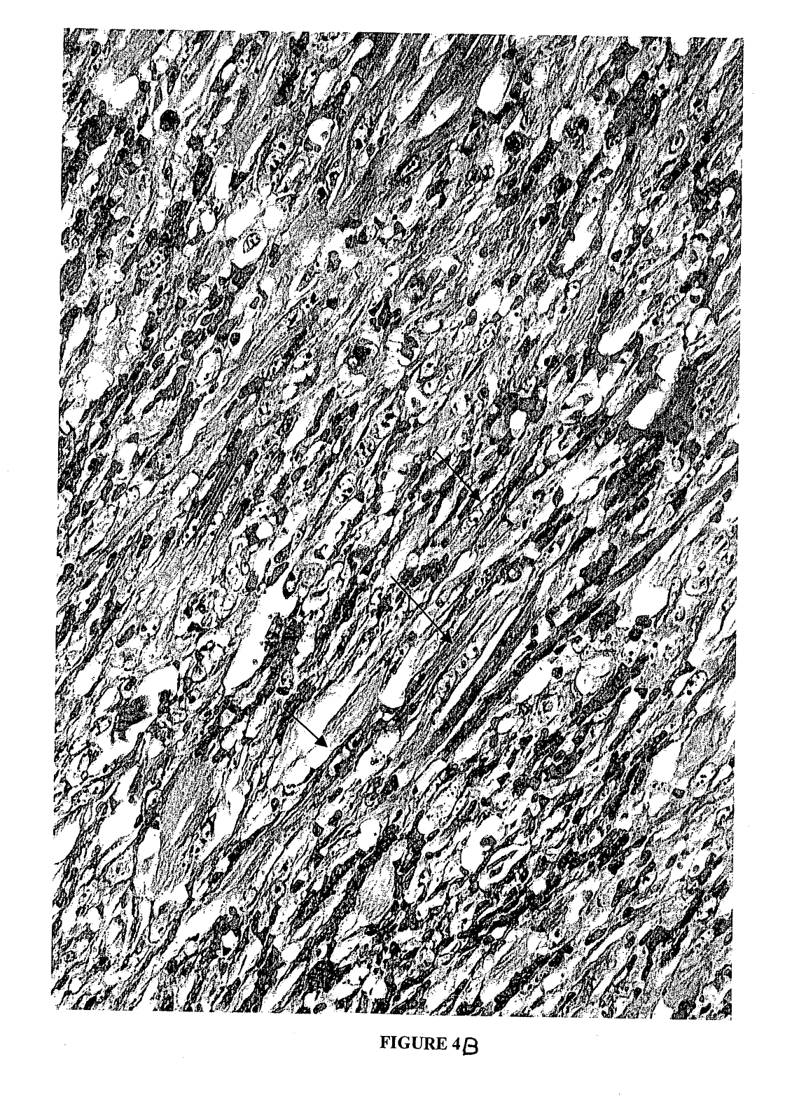

Approximately 300×10⁶ cells were transplanted using multiple injections into the left ventricular wall of the patient. Five days after injection the patient died and his heart was retrieved, fixed and sectioned. Surviving autologous skeletal muscle cells were identified in heavily scarred tissue of the heart by trichrome staining (FIGS. 4A and 4B). Myofiber structures were identified within the transplant region by the red trichrome stain characteristic of cardiac and skeletal muscle as opposed to the blue stain associated with fibroblasts and collagen of the scar (FIGS. 4A and 4B). The arrows in FIG. 4A indicate multiple cell deposits from multiple injections. As can be seen from FIG. 4A, the cell deposits remain closely centered around the injection site. At higher magnification (FIG. 4B), early fusion of the injected cells to form myotubes can be seen (see arrows).

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method of cell delivery, the method comprising steps of:
providing at least one isolated cell;
providing a needle with a side port;
injecting the cell through the needle into an organ.

2. The method of claim 1 further comprising step of sealing the injection site.

3. The method of claim 2, wherein the injection site is sealed with a cyanoacrylate tissue adhesive or fibrin sealant.

4. The method of claim 2, wherein the injection site is sealed with a film.

5. The method of claim 4, wherein the film is Seprafilm.

6. The method of claim 1, wherein the cell is provided with a carrier that aids in retention of cells at injection site.

7. The method of claim 1, wherein the carrier is selected from the group consisting of extracellullar matrix proteins, elastin, collagen, gelatin, fibrin, methylcellulose, agarose, hyaluronic acid, and alginate.

8. The method of cell delivery of claim 1, wherein the cell is a myocyte.

9. The method of cell delivery of claim 1, wherein the cell is a myoblast.

10. The method of cell delivery of claim 1, wherein the cell is a skeletal myocyte.

11. The method of cell delivery of claim 1, wherein the cell is a skeletal myoblast.

12. The method of cell delivery of claim 1, wherein the cell is a cardiac myocyte.

13. The method of cell delivery of claim 1, wherein the cell is a stem cell.

14. The method of cell delivery of claim 1, wherein the cell is derived from a stem cells.

15. The method of cell delivery of claim 1, wherein the cell is neuronal cell.

16. The method of cell delivery of claim 1, wherein the cell is a pancreatic islet cell.

17. The method of cell delivery of claim 1, wherein the cell is a hepatic cell.

18. The method of cell delivery of claim 1, wherein the cell is a renal cell.

19. The method of cell delivery of claim 1, wherein the cell is a pancreatic cell.

20. The method of cell delivery of claim 1, wherein the cell has been modified to mask cell surface antigens capable of causing a T-lymphocyte-mediated response upon transplantation in a recipient.

21. The method of cell delivery of claim 20, wherein the cell surface antigens are MHC molecules.

22. The method of cell delivery of claim 21, wherein the MHC molecules are MHC class I molecules.

23. The method of cell delivery of claim 20, wherein the cell surface antigens are masked with an antibody or fragment thereof.

24. The method of cell delivery of claim 20, wherein the cell surface antigens are masked with F(ab′)₂ fragments of antibodies.

25. The method of cell delivery of claim 20, wherein the cells surface antigens are masked with soluble T-cell receptor protein fragments.

26. The method of cell delivery of claim 1, wherein the organ is a heart.

27. The method of cell delivery of claim 1, wherein the cell is injected into the myocardium of a heart.

28. The method of cell delivery of claim 1, wherein the organ is a solid organ.

29. The method of cell delivery of claim 1, wherein the organ is selected from the group consisting of brain, liver, heart, pancreas, spleen, kidney, thyroid, prostate, and skeletal muscle.

30. The method of cell delivery of claim 1, wherein the needle has more than one side release port.

31. The method of cell delivery of claim 1, wherein the needle has a closed end.

32. The method of cell delivery of claim 1, wherein the needle is a Whitacre needle.

33. The method of cell delivery of claim 1, wherein the needle is a 25 gauge Whitacre needle.

34. The method of cell delivery of claim 1, wherein the needle is a 3½ inch, 25 gauge Whitacre needle.

35. The method of cell delivery of claim 1, wherein the needle is a 25 gauge needle.

36. The method of cell delivery of claim 1, wherein the needle is of a gauge between 20 and 25.

37. The method of cell delivery of claim 1, wherein the injection is performed during surgery so that the injection is not transdermal.

38. A method of treating a condition characterized by damage to cardiac tissue, the method comprising steps of:
providing a patient suffering from a condition characterized by damage to cardiac tissue;
providing skeletal myoblast cells;
providing a side release needle; and
injecting the cells using the needle into damaged cardiac tissue so as to treat the cardiac condition.

39. The method of claim 38, wherein the step of providing skeletal myoblast cells comprises providing a mixture of skeletal myobalst cells and fibroblasts.

40. A method of delivering an agent into solid tissue, the method comprising steps of:
providing an agent;
providing a needle with a side port; and
injecting the agent through the needle into solid tissue.

41. The method of claim 40, wherein the agent is a drug.

42. The method of claim 40, wherein the agent is a small molecule

43. The method of claim 40, wherein the agent is a protein.

44. The method of claim 40, wherein the agent is a peptide.

45. The method of claim 40, wherein the agent is a polynucleotide.

46. The method of claim 40, wherein the agent is a virus.

47. The method of claim 46, wherein the genome of the virus has been altered.

48. The method of claim 40, wherein the tissue is a neoplastic growth.

49. The method of claim 40, wherein the tissue is a malignant tumor.

50. The method of claim 40, wherein the tissue is a benign tumor.

51. A method of cell delivery, the method comprising steps of:
providing at least one isolated cell;
providing a needle with a side port;
injecting the cell through the needle into an organ at a depth at least 1 inch; and
allowing the needle to remain in the injection site for at least 30 seconds before removal.

52. The method of claim 51, wherein the cell is provided with a carrier that aid in retention of cells at injection site.

53. The method of claim 52 wherein the carrier is selected from the group consisting of extracellular matrix proteins, elastin, collagen, gelatin, fibrin, methylcellulos, agarose, alginate, and hyaluronic acid.

54. The method of claim 51 further comprising step of sealing the injection site with a tissue adhesive or film.

55. A kit comprising a needle with a closed end and a side opening and at least one cell for transplantation into a recipient.

56. The kit of claim 55, wherein the needle is sterile and the kit is package to maintain its sterility.

57. The kit of claim 55, wherein the at least one cell is provided in a carrier.

58. The kit of claim 57, wherein the carrier is selected from the group consisting of extracellular matrix proteins, elastin, collagen, gelatin, fibrin, methylcellulose, agarose, alginate, and hyaluronic acid.

59. The kit of claim 55 further comprising a tissue adhesive.

60. The kit of claim 55 further comprising a sealing film.

61. The kit of claim 55 further comprising a fibrin sealant.
